(19) United States
(12) Patent Application Publication
(10) Pub. No.: US 2012/0109261 A1
(43) Pub. Date: May 3, 2012

(54) PROTECTING AN IMPLANTABLE MEDICAL DEVICE FROM EFFECTS CAUSED BY AN INTERFERING RADIATION FIELD

(75) Inventors: Christopher C. Stancer, Prescott, WI (US); Steven D. Goedeke, Forest Lake, MN (US); Michael E. Nowak, Andover, MN (US)

(73) Assignee: MEDTRONIC, INC., Minneapolis, MN (US)

(21) Appl. No.: 13/098,164

(22) Filed: Apr. 29, 2011

Related U.S. Application Data
(60) Provisional application No. 61/408,302, filed on Oct. 29, 2010.

(51) Int. Cl. A61N 1/36

(52) U.S. Cl. 607/60

(57) ABSTRACT

Techniques are described for protecting an implantable medical device (IMD) from effects caused by interfering radiated fields. An IMD incorporating these techniques may include a telemetry conduction path that includes a first end electrically coupled to a telemetry antenna and a second end electrically coupled to a telemetry circuit disposed within a housing of the IMD. The IMD may further include a stub filter electrically coupled to the telemetry conduction path and configured to attenuate an interfering signal induced in the telemetry conduction path. The stub filter may include a dielectric and a conductor disposed within the dielectric. The conductor may include a first end electrically coupled to the telemetry conduction path and a second end configured in an open circuit configuration. The conductor may have an electrical length approximately equal to one-quarter wavelength of the interfering signal when propagating through the stub filter.

### RELATED APPLICATIONS

This application claims the benefit of U.S. Provisional Application No. 61/408,302, filed Oct. 29, 2010, the entire content of which is incorporated herein by reference.

### TECHNICAL FIELD

The disclosure relates to implantable medical devices (IMDs), and more particularly, to controlling effects caused by exposure of an IMD to an interfering radiation field.

### BACKGROUND

A variety of implantable medical devices (IMDs) exist that provide monitoring and/or therapeutic capabilities for a patient. Examples IMDs include implantable cardiac pacemakers, cardioverters, defibrillators, neurostimulators, muscle stimulators, and various other types of implantable tissue, organ and nerve stimulators and/or sensors. IMDs may use radio frequency (RF) telemetry to communicate with devices external to or implanted within a patient. For example, an IMD may utilize RF telemetry techniques to communicate with an external programming device, an external monitoring device, or any other device attached to a patient or located proximate to a patient. As another example, an IMD may utilize RF telemetry techniques to communicate with another implanted device, e.g., as part of an intra-body communications network. The information exchanged via RF telemetry techniques may include physiological data acquired by the IMD, information related to therapies delivered by the IMD, and information related to the operational status of the IMD. The IMD may also receive information from a programmer, such as configuration information that may be used to configure a therapy to be provided to the patient.

An IMD may be exposed to electromagnetic interference (EMI) for any of a number of reasons. Certain types of medical procedures may need to be performed on a patient within whom the IMD is implanted for purposes of diagnostics or therapy. For example, the patient may need to have a magnetic resonance imaging (MRI) scan, a computed tomography (CT) scan, electrocautery, diathermy or other medical procedure that produces a magnetic field, electromagnetic field, electric field or other type of electromagnetic energy.

The electromagnetic energy produced by such medical procedures may interfere with the operation of the IMD. For example, the electromagnetic energy may induce a current in one or more components within the telemetry system of the IMD, which may interfere with the operation of the internal circuitry within the IMD and/or alter the delivery of therapy by the IMD.

### SUMMARY

This disclosure is directed to an implantable telemetry system that includes a stub filter configured to attenuate an interfering signal induced within the telemetry system by external radiation fields. The implantable telemetry system may be used within an implantable medical device. The stub filter is electrically coupled to a telemetry conduction path situated between a telemetry antenna and a telemetry circuit. The stub filter may be configured to attenuate an interfering signal of a particular frequency or range of frequencies induced within the telemetry system. The interfering signal may be, in some examples, an interfering signal associated with a magnetic resonance imaging (MRI) scan. The stub filter may receive an incident wave associated with the interfering signal and generate a reflected wave that destructively interferes with the incident wave to generate a filtered wave. The resulting wave may have frequency components attributable to the interfering signal that are substantially reduced and/or eliminated. In this manner, the stub filter may reduce the interference caused by an external radiation field within a device in which the telemetry system is operating.

In one aspect, this disclosure is directed to an IMD that includes a telemetry conduction path that includes a first end electrically coupled to a telemetry antenna and a second end electrically coupled to a telemetry circuit disposed within a housing of the IMD. The IMD further includes a stub filter electrically coupled to the telemetry conduction path and configured to attenuate an interfering signal induced in the telemetry conduction path. The stub filter includes a dielectric and a conductor disposed within the dielectric. The conductor includes a first end electrically coupled to the telemetry conduction path and a second end configured in an open circuit configuration. The conductor has an electrical length approximately equal to one-quarter of the wavelength of the interfering signal when propagating through the stub filter.

In another aspect, this disclosure is directed to a method that includes attenuating, with a stub filter, an interfering signal induced in a telemetry conduction path that includes a first end electrically coupled to a telemetry antenna and a second end electrically coupled to a telemetry circuit disposed within a housing of the implantable medical device. The stub filter is electrically coupled to the telemetry conduction path. The stub filter includes a dielectric and a conductor disposed within the dielectric. The conductor includes a first end electrically coupled to the telemetry conduction path and a second end configured in an open circuit configuration. The conductor has an electrical length approximately equal to one-quarter of the wavelength of the interfering signal when propagating through the stub filter.

In another aspect, this disclosure is directed to an apparatus that includes a telemetry conduction path that includes a first end electrically coupled to a telemetry antenna and a second end electrically coupled to a telemetry circuit disposed within a housing of the implantable medical device. The apparatus further includes means for attenuating, with a stub filter electrically coupled to the telemetry conduction path, an interfering signal induced in the telemetry conduction path. The stub filter includes a dielectric and a conductor disposed within the dielectric. The conductor includes a first end electrically coupled to the telemetry conduction path and a second end configured in an open circuit configuration. The conductor has an electrical length approximately equal to one-quarter of the wavelength of the interfering signal when propagating through the stub filter.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a block diagram illustrating an example implantable telemetry system that implements RF interference attenuation techniques and may be used within an implantable medical device (IMD) according to this disclosure.

FIG. 2 is a conceptual diagram illustrating the propagation of an interfering signal through an example telemetry conduction path and stub filter configuration according to this disclosure.

FIG. 3 is a conceptual diagram illustrating destructive interference effects that occur in the telemetry conduction path and stub filter configuration of FIG. 2.

FIG. 4 is a conceptual diagram illustrating the change in wavelength produced by a wave propagating between different transmission mediums according to this disclosure.

FIG. 5 is a block diagram illustrating an example telemetry conduction path that may be utilized in the implantable telemetry system of FIG. 1 according to this disclosure.

FIG. 6 is a block diagram illustrating an example telemetry conduction path that may be utilized in the implantable telemetry system of FIG. 1 according to this disclosure.

FIG. 7 is a conceptual diagram illustrating an example stub filter that may be utilized in the implantable telemetry system of FIG. 1 according to this disclosure.

FIG. 8 is a conceptual diagram illustrating another example stub filter that may be utilized in the implantable telemetry system of FIG. 1 according to this disclosure.

FIG. 9 is a flow diagram illustrating an example technique for attenuating an interfering signal within an implantable telemetry system according to this disclosure.

FIG. 10 is a flow diagram illustrating another example technique for attenuating an interfering signal within an implantable telemetry system according to this disclosure.

FIG. 11 is a conceptual diagram illustrating an example therapy system that may utilize the implantable telemetry system of FIG. 1 according to this disclosure.

FIG. 12 is a conceptual diagram illustrating the IMD and leads of the example therapy system of FIG. 11 in greater detail.

FIG. 13 is a block diagram illustrating an example configuration of the IMD in the therapy system of FIG. 11 including an example RF interference attenuating telemetry system according to this disclosure.

### DETAILED DESCRIPTION

This disclosure is directed to an implantable telemetry system that includes a stub filter configured to attenuate an interfering signal induced with the telemetry system by external radiation fields. The stub filter may be configured to attenuate an interfering signal of a particular frequency or range of frequencies induced within the telemetry system. The interfering signal may be, in some examples, an interfering signal associated with a magnetic resonance imaging (MRI) scan. The stub filter may receive an incident wave associated with the interfering signal and generate a reflected wave that destructively interferes with the incident wave to generate a filtered wave. The resulting wave may have frequency components attributable to the interfering signal that are substantially reduced and/or eliminated. In this manner, the stub filter may prevent an external radiation field from interfering with the operation of the internal circuitry of the device in which the telemetry system is operating.

In some examples, the stub filter may include a conductor disposed within a dielectric. The conductor may have an electrical length approximately equal to one-quarter of the wavelength of the signal to be attenuated (e.g., the interfering signal). Thus, stub filter may form a one-quarter wavelength stub filter. As used herein, the length of stub filter may refer to the length of the transmission medium in the stub filter, e.g., the conductor within the stub filter. The term electrical length may refer to the length of the transmission medium in the stub filter expressed as a number of wavelengths of the interfering signal when propagating through the transmission medium. In contrast, the term physical length, as used herein, may refer to the length of the transmission medium in the stub filter expressed in units of length independent of the wavelength of the interfering signal. The interfering signal may be a signal having a frequency which the stub filter is designed to attenuate, e.g., a 45 megahertz (MHz) signal produced by a 1.0 Tesla (T) MRI scanner, a 64 MHz signal produced by a 1.5 T MRI scanner, or a 128 MHz signal produced by a 3.0 T MRI scanner. In some examples, the wavelength of the interfering signal when propagating through the transmission medium may be less than the wavelength of the interfering signal when propagating through free space or air. In additional examples, the wavelength of the interfering signal when propagating through the transmission medium may be less than the wavelength of the interfering signal when propagating through the telemetry conduction path.

In some examples, the stub filter may include a dielectric having a high dielectric constant value. The high dielectric constant value may allow the physical length of the conductor in the stub filter to be reduced so that the stub filter can fit within the connector block and/or housing of an implantable medical device (IMD) implementing the telemetry system of this disclosure.

The dielectric constant of a dielectric may be dependent on temperature. Dielectrics that are designed to have a high dielectric constant value may experience increased sensitivity to temperature fluctuations. In some examples, a stub filter according to this disclosure may operate in an environment with sufficient temperature stability to prevent large fluctuations in the dielectric constant even in cases where the dielectric has a high dielectric constant value, e.g., a dielectric constant value greater than 9000. Such an environment may be, for example, the patient in which an IMD including the stub filter is implanted.

Many IMDs that provide therapy via electrodes include an electrode feedthrough assembly which includes a feedthrough capacitor configured to route RF interference above a particular frequency to the housing of the IMD. An IMD telemetry system may also include a feedthrough assembly positioned between a telemetry antenna situated outside of the housing of the IMD and other telemetry circuitry situated inside of the housing. Because the telemetry system may communicate using telemetry signals within the RF frequency range (e.g., 400 MHz), a feedthrough capacitor in the telemetry feedthrough assembly would suppress the RF telemetry signal in addition to suppressing unwanted RF interference. Thus, unlike the electrode feedthrough assembly, the telemetry feedthrough assembly may be designed to not include a feedthrough capacitor. The stub filter RF attenuation techniques in this disclosure, however, may be used to suppress or attenuate particular frequencies from reaching the internal circuitry of an IMD while still allowing RF telemetry signals to reach the internal circuitry. In this manner, a stub filter designed in accordance with this disclosure may act as a notch filter with a stop band that occupies the frequency of the interfering signal and a pass band that occupies the frequency at which telemetry communications take place.

As indicated above, this disclosure describes a one-quarter wavelength open circuit stub filter to generate a reflected waveform that attenuates or cancels an interfering radiating field. The interfering radiating field may, for example, be a radiating field generated by an MRI scanner. The techniques of this disclosure may, however, be used to reduce and/or eliminate the effect of other interfering radiating fields, such as interfering radiating fields generated by any medical or non-medical device.

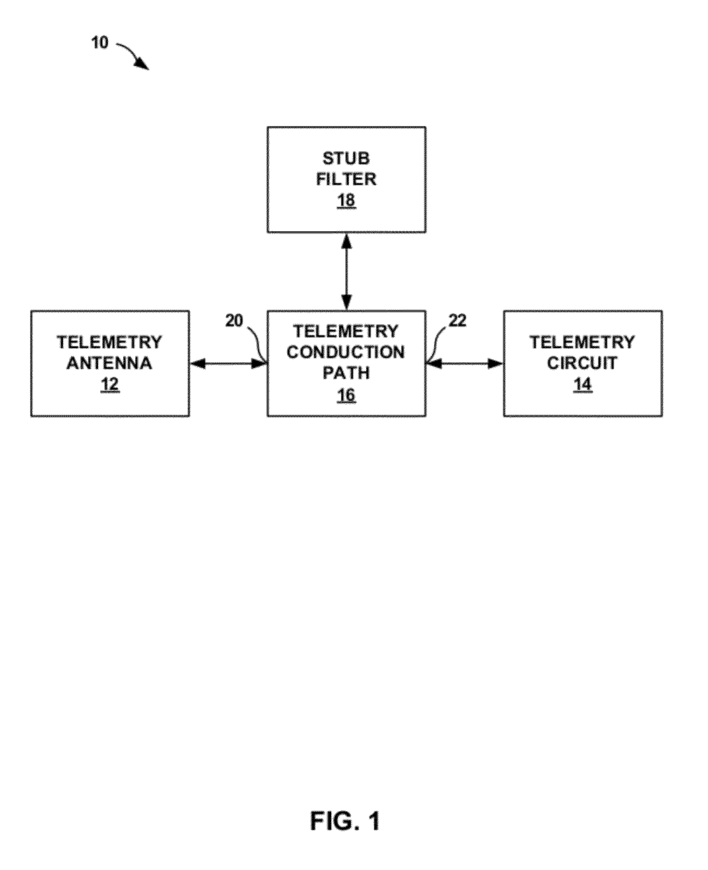

FIG. 1 is a block diagram illustrating an example implantable telemetry system 10 that implements RF interference attenuation techniques and may be used within an (IMD according to this disclosure. Telemetry system 10 is configured to provide remote communications between an implantable medical device and another device via RF telemetry techniques. As used herein, RF telemetry techniques may refer to wireless telemetry techniques or other non-inductive telemetry techniques. Telemetry system 10 may form part of an IMD. According to this disclosure, telemetry system 10 is configured to protect telemetry circuit 12 and/or other components within an IMD housing from effects caused by RF interference. Telemetry system 10 includes a telemetry antenna 12, a telemetry circuit 14, a telemetry conduction path 16 and a stub filter 18.

Telemetry antenna 12 is configured to act as a transmission antenna and/or a receiver antenna for telemetry system 10. Telemetry antenna 12 is electrically coupled to telemetry conduction path 16 at end 20.

When acting as a receiver antenna, telemetry antenna 12 is configured to receive an RF telemetry signal and to convert the RF telemetry signal into a receive signal. In some examples, the RF telemetry signal may include electromagnetic waves transmitted via RF telemetry techniques. In further examples, the receive signal may include electrical current waves. The receive signal, in some examples, may be a modulated signal that includes a data signal modulated onto a carrier wave. Telemetry antenna 12 may provide the receive signal to telemetry conduction path 16 for transport to telemetry circuit 14.

When acting as a transmission antenna, telemetry antenna 12 is configured to receive a transmit signal from telemetry conduction path 16 and to convert the transmit signal into an RF telemetry signal for transmission to another device. In some examples, the transmit signal may include electrical current waves. The transmit signal may, in some examples, be a modulated signal that includes a data signal modulated onto a carrier wave. In further examples, the RF telemetry signal may include electromagnetic waves generated according to RF telemetry techniques.

Telemetry antenna 12 may be any type of antenna configured to transmit and receive RF telemetry signals. For example, telemetry antenna 12 may take the form of a dipole antenna, a microstrip antenna, a monopole antenna, or any other type of antenna. In some examples, telemetry antenna 12 may be configured to transmit and receive RF telemetry signals within a frequency range of 300 megahertz (MHz) to 500 MHz, and more particularly within in a frequency range of 402 MHz to 405 MHz, such as, e.g., the Medical Implant Communication Service (MICS) frequency band. In some examples, telemetry antenna 12 may be configured to transmit and receive RF telemetry signals at a frequency of approximately 400 MHz.

Telemetry circuit 14 is configured to act as a telemetry receiver, a telemetry transmitter, and/or a telemetry transceiver for telemetry system 10. Telemetry circuit 14 is electrically coupled to telemetry conduction path 16 at end 22.

When acting as a telemetry receiver, telemetry circuit 14 is configured to receive a receive signal from telemetry conduction path 16, and to convert the receive signal into a data signal for use by other components within the device in which telemetry system 10 operates. In some examples, the receive signal may be a modulated signal that includes a data signal modulated onto a carrier wave. In such examples, telemetry circuit 14 may be configured to demodulate the telemetry receive signal to produce the data signal. The data signal may be a demodulated signal that includes the data signal component of the receive signal with the carrier signal removed.

When acting as a telemetry transmitter, telemetry circuit 14 is configured to receive a data signal from another component within the device in which telemetry system 10 operates, and to convert the data signal into a transmit signal. Telemetry circuit 14 may provide the transmit signal to telemetry conduction path 16 for transport to telemetry antenna 12. In some examples, telemetry circuit 14 may be configured to modulate the data signal onto a carrier wave to produce the transmit signal. In such examples, the transmit signal may include a data signal component modulated onto a carrier wave.

Besides modulation and demodulation of telemetry signals, telemetry circuit 14 may also perform other telemetry communications functions. Telemetry circuit 14 may be implemented as a controller, a processor, an application specific integrated circuit (ASIC), discrete circuitry, an integrated circuit, or any combination thereof.

Telemetry conduction path 16 is configured to transfer signals between telemetry antenna 12 and telemetry circuit 14. For example, telemetry conduction path 16 may receive a receive signal from telemetry antenna 12 and provide the receive signal to telemetry circuit 14 for further processing. As another example, telemetry conduction path 16 may receive a transmit signal from telemetry circuit 14 and provide the transmit signal to telemetry antenna 12 for telemetry transmission. Telemetry conduction path 16 includes end 20 electrically coupled to telemetry antenna 12, and end 22 electrically coupled to telemetry circuit 14. Telemetry conduction path 16 is electrically coupled to stub filter 18 at a location between end 20 and end 22 of telemetry conduction path 16.

Telemetry conduction path 16 includes a main line conductive path disposed between telemetry antenna 12 and telemetry circuit 14. The main line conductive path is configured to electrically carry the transmit and receive signals between telemetry antenna 12 and telemetry circuit 14. In some examples, the main line conductive path may include a single main line conductor having a first end 20 electrically coupled to telemetry antenna 12 and a second end 22 electrically coupled to telemetry circuit 14.

In further examples, the main line conductive path may include any number of intervening components and/or circuitry between telemetry antenna 12 and telemetry circuit 14. In such examples, the main line conductive path may include multiple main line conductors each configured to electrically couple two intervening components to each other or to electrically couple an intervening component to one of telemetry antenna 12 and telemetry circuit 14. Each intervening component may be configured to transfer the transmit or receive signal from a first main line conductor to a second main line conductor both of which are electrically coupled to the intervening component. In such examples, the main line conductors together with the intervening components may form the main line conductive path and operate to carry the transmit and receive signals between telemetry antenna 12 and telemetry circuit 14.

In additional examples, telemetry conduction path 16 may include a secondary conductive path that is regulated at an RF ground potential. The secondary conductive path, in some examples, may be a RF ground plane for a printed circuit board. The secondary conductive pathway may, but need not, span the entire distance between telemetry antenna 12 and telemetry circuit 14. In addition, the secondary conductive pathway may include the same or a different set of intervening components as the main line conductor as well as no intervening components at all.

Similar to the main line conductive path, the secondary conductive path may include a single secondary conductor or multiple secondary conductors electrically coupled between intervening components. The mainline conductors and the secondary conductors may form one or more two-conductor transmission lines and operate together to propagate electrical waves (e.g., current or voltage waves) between telemetry antenna 12 and telemetry circuit 14.

The main line conductors and the secondary conductors may be implemented as copper wires, conductive traces, laser ribbon bond, interconnect ribbons, or any other type of conductor configured to electrically couple different components together.

Stub filter 18 is configured to attenuate an interfering signal induced within telemetry system 10. Stub filter 18 may be further configured to cause a reflected version of the interfering signal to propagate through the stub filter, and to cause the reflected version of the interfering signal to destructively interfere with the interfering signal at the junction between stub filter 18 and telemetry conduction path 16 to produce a filtered signal. Stub filter 18 and telemetry conduction path 16 may provide the filtered signal to telemetry circuit 14. The frequency components attributable to the interfering signal may be substantially attenuated, reduced, and/or eliminated in the filtered signal. Stub filter 18 is electrically coupled to telemetry conduction path 16 to form a junction or connection point between stub filter 18 and telemetry conduction path 16.

Stub filter 18 may include a conductor having a first end electrically coupled to telemetry conduction path 16 and a second end that is configured in an open circuit configuration. The conductor may be referred to herein as a “main line conductor.” In some examples, the first end of conductor may be electrically coupled to the main line conductive path (e.g., a main line conductor) of telemetry conduction path 16.

The conductor of stub filter 18 may take on a variety of shapes and forms. In some examples, the conductor may be an elongated conductor having a first end and a second end. In further examples, the conductor may be shaped substantially in a straight line. In additional examples, the conductor may include curves or bends, such as, e.g., a coiled or helical conductor.

Stub filter 18 may also include a dielectric within which the conductor is formed or otherwise disposed. In some examples, the dielectric may span the entire length of the main line conductor. In additional examples, the dielectric may surround the conductor.

As used herein, an open circuit configuration may refer to a configuration where the second end of the conductor is not electrically coupled to a secondary conductor, e.g., the second end of the conductor is electrically isolated from the secondary conductor. In contrast, a closed circuit configuration may refer to a configuration where the second end of the conductor is electrically coupled to the secondary conductor.

The physical length of stub filter 18 may be selected such that stub filter 18 may fit completely within the connector block and/or the housing of an IMD that includes implantable telemetry system 10. For example, stub filter 18 may be configured such that the length of the main line conductor is less than approximately 0.5 inches.

In some examples, in order to achieve the desired physical length for stub filter 18, a high dielectric constant value may be selected for the dielectric in stub filter 18. For example, the dielectric may have a dielectric constant that is at least approximately 500, and more particularly, at least approximately 1000, and more particularly, at least approximately 5000, and more particularly, at least approximately 9000. In free space, one-quarter of a wavelength of a 64 MHz wave is over one meter long. In a typical coaxial cable with a dielectric constant of 2.29, one-quarter of a wavelength of a 64 MHz wave is over one-half of a meter long. These dimensions are too long for many implantable medical device implementations. Thus, by using a dielectric with a high dielectric constant, as described in this disclosure, the physical length of stub filter 18 may be reduced to fit within an IMD.

In some examples, stub filter 18 may further include a secondary conductor that is separated from the main line conductor by a dielectric. The secondary conductor may be electrically coupled to the secondary conductive path (e.g., a secondary conductor) of telemetry conduction path 16. In such examples, the main line conductor and the secondary conductor may form a transmission line. Because the second end of the main line conductor is in an open circuit configuration, the second end of the main line conductor is not electrically coupled to the secondary conductor. Such a configuration may be referred to herein as an “open circuit terminated transmission line.”

The operation of telemetry system 10 will now be described. Telemetry system 10 is placed within an environment where RF interference is present. For example, telemetry system 10 may be implemented within an IMD implanted in a patient who is undergoing an MRI scan. The MRI scan may produce RF interference (e.g., electromagnetic interference) at a frequency of 64 MHz for example.

The RF interference induces an interfering signal within telemetry system 10. The RF interference may cause an interfering signal to be induced within telemetry antenna 12, within telemetry conduction path 16, or within both telemetry antenna 12 and telemetry conduction path 16. In some examples, the induced signal may take the form of an induced current.

Telemetry conduction path 16 carries the interfering signal to stub filter 18. Stub filter 18 receives the interfering signal and attenuates the interfering signal to generate a filtered signal. The frequency components attributable to the interfering signal in the resulting filtered signal may be substantially attenuated and/or eliminated. In this manner, stub filter 18 may protect telemetry circuit 14 as well as other components in an IMD containing telemetry system 10 from effects caused by RF interference.

Telemetry system 10 may be configured to receive and transmit telemetry signals when telemetry system 10 is not within an environment where RF interference is present. In cases where telemetry system 10 is receiving a telemetry signal, telemetry antenna 12 receives an RF telemetry signal and converts the RF telemetry signal into a receive signal. Telemetry conduction path 16 receives the receive signal at end 20 from telemetry antenna 12 and provides the receive signal to telemetry circuit 14, via end 22, for further processing. The receive signal may have frequency components that are different than the frequency components of the interfering signal. For example, the frequency components in the receive signal may correspond to the frequency at which telemetry communications take place. Thus, while telemetry conduction path 16 is carrying the receive signal, stub filter 18 allows frequency components attributable to the receive signal to pass between telemetry antenna 12 and telemetry circuit 14. Telemetry circuit 14 receives the receive signal from telemetry conduction path 16, and converts the receive signal into a data signal for use by other components within the device in which telemetry system 10 operates.

In cases where telemetry system 10 is transmitting a telemetry signal, telemetry circuit 14 receives a data signal from another component within the device in which telemetry system 10 operates, and converts the data signal into a transmit signal. Telemetry conduction path 16 receives the transmit signal at end 22 from telemetry circuit 14 and provides the transmit signal to telemetry antenna 12, via end 20, for telemetry transmission. Similar to the receive signal, the transmit signal may have frequency components that correspond to the frequency at which telemetry communications take place. Thus, while telemetry conduction path 16 is carrying the transmit signal, stub filter 18 allows frequency components attributable to the transmit signal to pass between telemetry circuit 14 and telemetry antenna 12. Telemetry antenna 12 receives the transmit signal from telemetry conduction path 16 and converts the transmit signal into an RF telemetry signal for transmission to another device.

As discussed in the examples above, stub filter 18 may be configured to attenuate frequency components attributable to an interfering signal of a particular frequency while allowing other frequency components attributable to telemetry transmit and receive signals to pass between telemetry antenna 12 and telemetry circuit 14 relatively unimpeded. In this manner, stub filter 18 may act as a notch filter with a stop band that occupies the frequency of the interfering signal and a pass band that occupies the frequency at which telemetry communications take place.

Implantable telemetry system 10 may be implemented within an IMD that includes circuitry enclosed within a hermetically-sealed housing and a connector block attached to the housing. According to a first example, the first end of the conductor of stub filter 18 is electrically coupled to telemetry conduction path 16 at a location external to the housing. According to a second example, the first end of the conductor of stub filter 18 is electrically coupled to telemetry conduction path 16 at a location within the connector block of the IMD.

According to a third example, the first end of the conductor of stub filter 18 is electrically coupled to telemetry conduction path 16 at a location in the interior of the housing. In a first implementation of the third example, telemetry conduction path 16 comprises an impedance matching circuit, and the first end of the conductor of stub filter 18 is electrically coupled to telemetry conduction path 16 at a location between the impedance matching circuit and telemetry circuit 14.

In a second implementation of the third example, telemetry conduction path 16 comprises an impedance matching circuit, and the first end of the conductor of stub filter 18 is electrically coupled to telemetry conduction path 16 at a location between telemetry antenna 12 and the impedance matching circuit. According to a first example of the second implementation, telemetry conduction path 16 may further comprise a protection circuit located between telemetry antenna 12 and the impedance matching circuit, the protection circuit being configured to protect telemetry circuit 14 from at least one type of interfering signal condition, and the first end of the conductor of stub filter 18 is electrically coupled to telemetry conduction path 16 at a location between telemetry antenna 12 and the protection circuit. According to a second example of the second implementation, telemetry conduction path 16 comprises a protection circuit located between telemetry antenna 12 and the impedance matching circuit, the protection circuit being configured to protect telemetry circuit 14 from at least one type of interfering signal condition, and the first end of the conductor of stub filter 18 is electrically coupled to telemetry conduction path 16 at a location between the protection circuit and the impedance matching circuit.

Although several examples and implementations have been described above for implantable telemetry system 10 of FIG. 1, it should be understood that this disclosure is not limited to such examples and implementations, and that the techniques may be applied in other implementations as well as in other environments having telemetry conduction paths that are susceptible to RF interference.

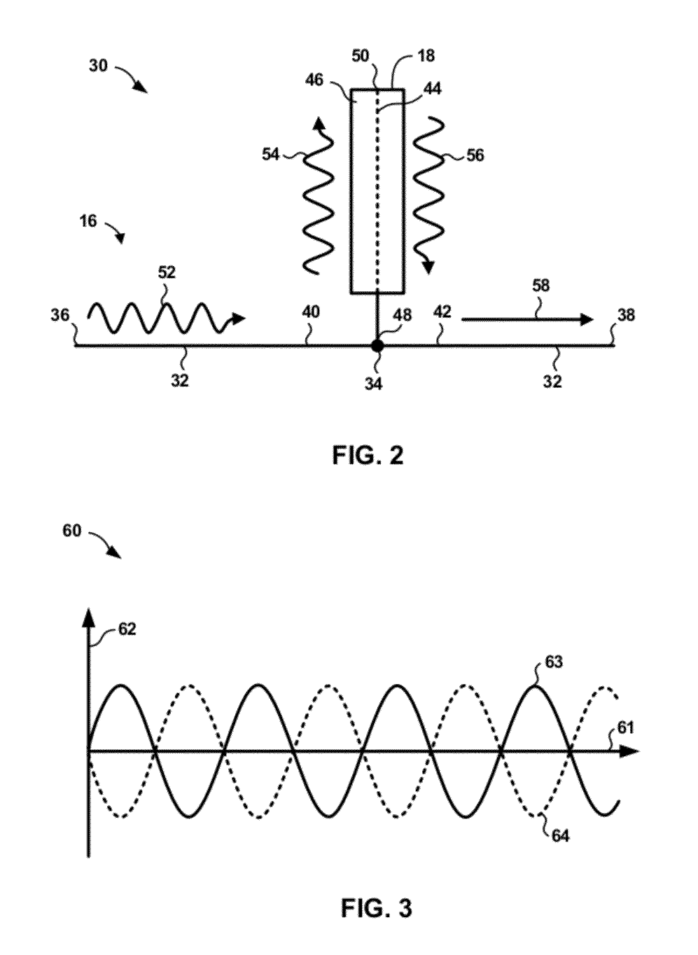

FIG. 2 is a conceptual diagram illustrating the propagation of an interfering signal through an example telemetry conduction path and stub filter configuration 30 according to this disclosure. Telemetry conduction path and stub filter configuration 30 may be utilized in the implantable telemetry system 10 of FIG. 1. Identically numbered components between FIGS. 1 and 2 may perform the same or similar functionality and be constructed from the same or similar components. Thus, in the interest of brevity and to avoid redundancy, these identically numbered components will not be described in further detail.

Telemetry conduction path and stub filter configuration 30 includes telemetry conduction path 16 and stub filter 18. Telemetry conduction path 16 includes main line conductive path 32 and junction 34. Main line conductive path 34 includes ends 36 and 38. Junction 34 is positioned between end 36 and end 38. In some examples, end 36 may correspond to end 20 in the telemetry system of FIG. 1 and be electrically coupled to a telemetry antenna. In additional examples, end 38 may correspond to end 22 in the telemetry system of FIG. 1 and be electrically coupled to a telemetry circuit. In further examples, one or both of ends 36 and 38 may be electrically coupled to respective intervening components in telemetry conduction path 16.

Main line conductive path 32 includes a distal portion 40 that is defined between end 36 and junction 34, and a proximal portion 42 that is defined between junction 34 and end 38. In this context distal and proximal are used in relation to telemetry circuit 14.

Stub filter 18 includes a conductor 44 and a dielectric 46. End 48 of conductor 44 is electrically coupled to main line conductive path 32 at junction 34. End 50 of conductor 44 is configured in an open circuit configuration. As indicated by the dotted line in FIG. 2, some or all of conductor 44 may be disposed within dielectric 46.

During operation, an incident wave 52 is either induced in distal portion 40 of main line conductive path 32 or received by distal portion 40 of main line conductive path 32 via end 36. Interfering signal 52 may have a particular frequency which stub filter 18 has been designed to attenuate. Interfering signal 52 propagates to junction 34. At junction 34, interfering signal 52 propagates down conductor from end 48 to end 50 as incident wave 54.

The open circuit end 50 of stub filter 18 causes a reflected wave 56 to propagate through conductor 48 towards junction 34. Reflected wave 56 may be substantially in-phase with incident wave 54 at end 50. In other words, the reflection coefficient of end 50 may be approximately equal to one. Because the length of stub filter 18 is approximately one-quarter of the wavelength of incident wave 54, reflected wave 56 may be approximately 180 degrees out-of-phase with incident wave 54 at junction 34. Thus, incident wave 54 and reflected wave 56 may destructively interfere with each other at junction 34 to produce filtered signal 58. The frequency components attributed to interfering signal 32 may be substantially attenuated and/or suppressed in filtered signal 58 as represented in FIG. 2 by a filtered waveform 58 of substantially zero magnitude. In some examples, filtered signal 58 may include frequency components attributed to interfering signal 32 that are reduced relative to interfering signal 52 but not completely suppressed. In this manner, the downstream circuitry is protected from an interfering signal 52 induced within and/or propagating through telemetry conduction path 16.

The conceptual diagram in FIG. 2 is provided merely to depict general concepts of this disclosure. As such, it is understood that conceptual diagram is not intended to be a mathematically or physically rigorous depiction of waveforms travelling through the telemetry conduction path and stub filter configuration 30 of FIG. 2. For example, the waveforms 52, 54 and 56 illustrated in FIG. 2 are illustrated as sinusoidal waveforms to convey that such waveforms are periodic in nature. It is understood, however, that the wavelengths of waveforms 52, 54 and 56 are not drawn to scale with respect to the telemetry conduction path and stub filter configuration 30 shown in FIG. 2. For example, stub filter 18 is a one-quarter wave stub filter. Thus, multiple wavelengths of each of incident wave 54 and reflected wave 56 would not be simultaneously present in stub filter 18 as shown in FIG. 2. Rather, approximately one-quarter of the wavelength of each of waveforms 54 and 56 would be present in stub filter 18.

FIG. 3 is a conceptual diagram 60 illustrating destructive interference effects that occur in the telemetry conduction path and stub filter configuration 30 of FIG. 2. Diagram 60 includes axes 61, 62, and waveforms 63, 64. Axis 61 represents a time axis increasing in time from left to right. Axis 62 represents the voltage at junction 34. Waveform 63 represents incident wave 54 at junction 34 and waveform 64 represents reflected wave 56 at junction 34.

As shown in FIG. 3, at every location along axis 61, waveforms 63 and 64 have magnitudes that are substantially equal, but opposite in polarity. Thus, at every instance in time, the sum of waveforms 63 and 64 is substantially zero. The sum of waveforms 63 and 64 corresponds to destructive interference between waveforms 63 and 64. Hence, the resulting composite waveform along axis 61 has substantially zero magnitude. The resulting composite waveform may correspond to filtered signal 58.

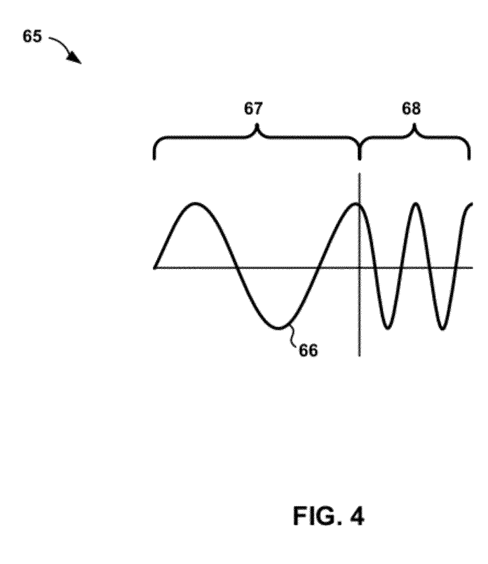

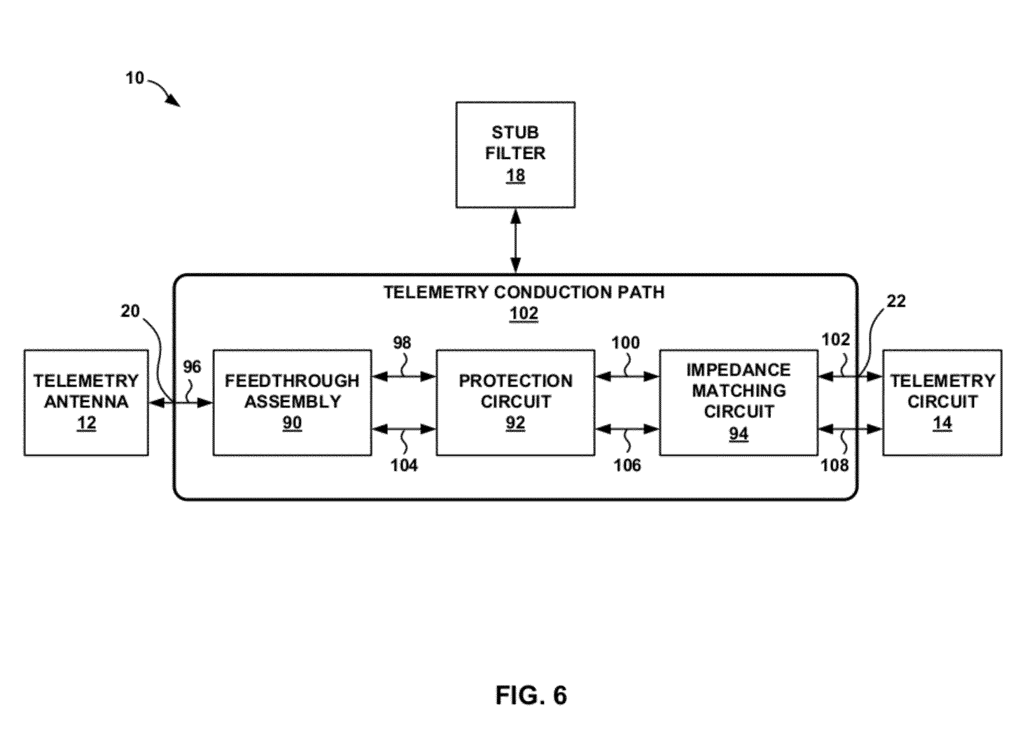

FIG. 4 is a conceptual diagram 65 illustrating the change in wavelength produced by a wave propagating between different transmission mediums according to this disclosure. As shown in diagram 65, a wave 66 propagates through transmission mediums 67 and 68. Transmission medium 67 corresponds to telemetry conduction path 16, and transmission medium 68 corresponds to stub filter 18. Because the dielectric constants are different between the transmission mediums, the wavelength of wave 66 changes between the different transmission mediums 67 and 68. In this example, stub filter 18 (i.e., transmission medium 68) reduces the wavelength of an interfering signal (i.e., wave 66) received from telemetry conduction path 16 (i.e., transmission medium 67). By reducing the wavelength of the interfering signal, a one-quarter wavelength stub filter may be designed to attenuate MRI interference while having a physical length small enough to fit within the connector block and/or housing of an IMD.

As already discussed above with respect to FIG. 2, it is understood that conceptual diagram in FIG. 4 is not intended to be a mathematically or physically rigorous depiction of waveforms travelling through telemetry conduction path 16 and stub filter 18 in this disclosure. For example, transmission medium 68 corresponds to a one-quarter wave stub filter 18. Thus, multiple wavelengths of waveform 66 would not be simultaneously present in stub filter 18 as shown in FIG. 2. Rather, approximately one-quarter of the wavelength of waveform 68 would be present in stub filter 18.

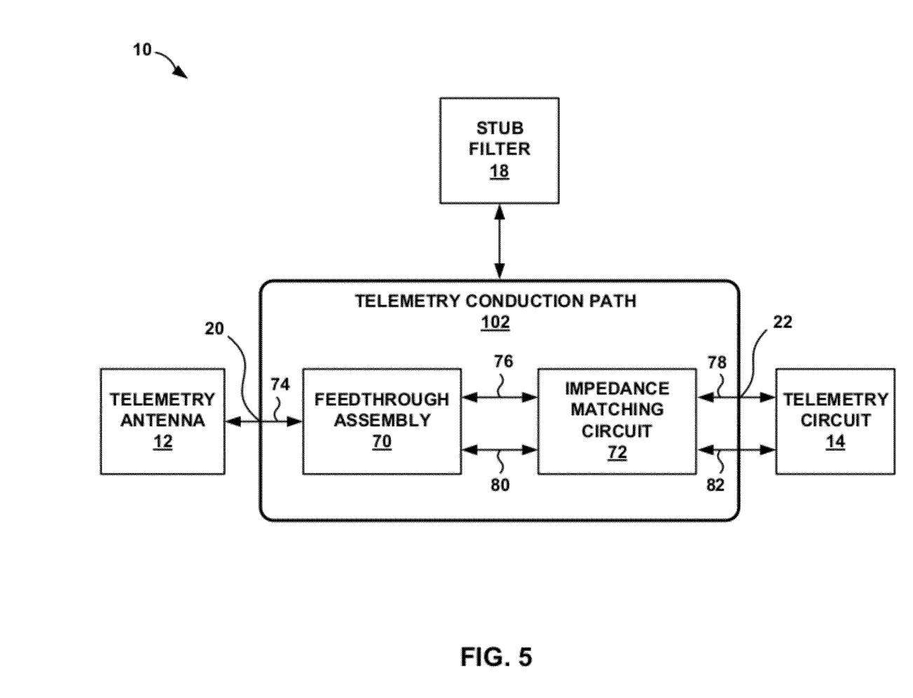

FIG. 5 is a block diagram illustrating an example telemetry conduction path 16 that may be utilized in the implantable telemetry system 10 of FIG. 1 according to this disclosure. Identically numbered components between FIGS. 1 and 5 may perform the same or similar functionality and be constructed from the same or similar components. Thus, in the interest of brevity and to avoid redundancy, these identically numbered components will not be described in further detail.

Telemetry conduction path 16 includes feedthrough assembly 70, impedance matching circuit 72, main line conductors 74, 76, 78, and secondary line conductors 80, 82. Main line conductors 74, 76, 78 may form a main line conductive path as described above with respect to FIG. 1. Secondary line conductors 80, 82 may form a secondary line conductive path as described above with respect to FIG. 1. In some examples, secondary line conductors 80, 82 may be electrically coupled to an RF ground. Feedthrough assembly 70 and impedance matching circuit 72 may each form an intervening component as described above with respect to FIG. 1. Although the example telemetry conduction path 16 in FIG. 5 includes two intervening components, in other examples, one or more additional intervening components may also be positioned between any of telemetry antenna 12, feedthrough assembly 70, impedance matching circuit 72, and telemetry circuit 14.

Feedthrough assembly 70 is configured to transfer receive and transmit signals between the outside and inside of a housing for a device in which implantable telemetry system 10 operates. When telemetry system 10 is acting as a receiver, feedthrough assembly 70 is configured to receive a receive signal from telemetry antenna 12 via conductor 74 and to provide the receive signal to impedance matching circuit 72 via conductors 76, 80. When telemetry system 10 is acting as a transmitter, feedthrough assembly 70 is configured to receive a transmit signal from impedance matching circuit 72 via conductors 76, 80 and to provide the transmit signal to telemetry antenna 12 via conductor 74.

In some examples, feedthrough assembly 70 may correspond to a conventional telemetry feedthrough assembly 70 within an IMD or an implantable cardiac defibrillator (ICD). For example, feedthrough assembly 70 may include a feedthrough conductor having a first end positioned outside of the housing of an IMD and a second end positioned inside of the housing. Feedthrough assembly 70 may be configured to allow signals having frequencies at which telemetry takes place to pass from the exterior of the housing to the interior of the housing. In such examples, feedthrough assembly 70 may not include a feedthrough capacitor. The exclusion of a feedthrough capacitor in such examples may prevent legitimate telemetry signals from being shunted to ground.

Impedance matching circuit 72 is configured to transfer receive and transmit signals between a first port 76, 80 and a second port 78, 82. The input impedance of the first port 76, 80 may be configured to match the impedance of telemetry antenna 12 and the input impedance of the second port 78, 82 may be configured to match the impedance of telemetry circuit 14. When telemetry system 10 is acting as a receiver, impedance matching circuit 72 is configured to receive a receive signal from feedthrough assembly 70 via conductors 76, 80 and to provide the receive signal to telemetry circuit 14 via conductors 78, 82. When telemetry system 10 is acting as a transmitter, impedance matching circuit 72 is configured to receive a transmit signal from telemetry circuit 14 via conductors 78, 82 and to provide the transmit signal to feedthrough assembly 70 via conductors 76, 80.

Stub filter 18 may be electrically coupled to telemetry conduction path 16 in one of several locations. For example, an end of the conductor in stub filter 18 may be electrically coupled to one of main line conductors 74, 76 and 78. When stub filter 18 includes a secondary conductor, an end of the secondary conductor may be electrically coupled to one of secondary line conductors 80, 82. In some examples, secondary line conductors 80, 82 may be electrically coupled to an RF ground. The RF ground may, in some examples, be capacitively coupled to the housing of the device in which implantable telemetry system 10 operates.

Feedthrough assembly 70 separates those components situated outside of the housing of a device in which telemetry system 10 is operating from those situated within the housing. Thus, telemetry antenna 12 is located outside of the housing while impedance matching circuit 72 and telemetry circuit 14 are located within the housing. When stub filter 18 is electrically coupled to conductor 74, stub filter 18 may reside completely outside of the housing of the device. When stub filter is electrically coupled to conductors 76, 78, stub filter 18 may reside completely within the housing of the device.

FIG. 6 is a block diagram illustrating an example telemetry conduction path 16 that may be utilized in the implantable telemetry system 10 of FIG. 1 according to this disclosure. Identically numbered components between FIGS. 1 and 6 may perform the same or similar functionality and be constructed from the same or similar components. Thus, in the interest of brevity and to avoid redundancy, these identically numbered components will not be described in further detail.

Telemetry conduction path 16 includes feedthrough assembly 90, protection circuit 92, impedance matching circuit 94, main line conductors 96, 98, 100, 102, and secondary line conductors 104, 106, 108. Main line conductors 96, 98, 100, 102 may form a main line conductive path as described above with respect to FIG. 1. Secondary conductors 104, 106, 108 may form a secondary line conductive path as described above with respect to FIG. 1. Feedthrough assembly 90, protection circuit 92, and impedance matching circuit 94 may each form an intervening component as described above with respect to FIG. 1. Although the example telemetry conduction path 16 in FIG. 6 includes three intervening components, in other examples, one or more additional intervening components may also be positioned between any of telemetry antenna 12, feedthrough assembly 90, protection circuit 92, impedance matching circuit 94, and telemetry circuit 14.

Feedthrough assembly 90 is configured to transfer receive and transmit signals between the outside and inside of a housing for a device in which implantable telemetry system 10 operates. When telemetry system 10 is acting as a receiver, feedthrough assembly 90 is configured to receive a receive signal from telemetry antenna 12 via conductor 96 and to provide the receive signal to protection circuit 92 via conductors 98, 104. When telemetry system 10 is acting as a transmitter, feedthrough assembly 90 is configured to receive a transmit signal from protection circuit 92 via conductors 98, 104 and to provide the transmit signal to telemetry antenna 12 via conductor 96.

In some examples, feedthrough assembly 90 may be configured to allow signals having frequencies at which telemetry takes place to pass from the exterior of the housing to the interior of the housing. In such examples, feedthrough assembly 90 may not include a feedthrough capacitor. The exclusion of a feedthrough capacitor in such examples may prevent legitimate telemetry signals from being shunted to ground.

Protection circuit 92 is configured to protect telemetry circuit 14 from at least one type of interference condition. For example, protection circuit 92 may provide high voltage protection functionality to protect telemetry circuit 14 from a high voltage interference condition. As another example, protection circuit 92 may provide RF limiting functionality to protect telemetry circuit 14 from a radio frequency interference condition. In some examples, protection circuit 92 may include a PIN limiter diode that protects telemetry circuit 14 from large external RF signals.

When telemetry system 10 is acting as a receiver, protection circuit 92 is configured to receive a receive signal from feedthrough assembly 90 via conductors 98, 104 and to provide the receive signal to impedance matching circuit 94 via conductors 100, 106. When telemetry system 10 is acting as a transmitter, protection circuit 92 is configured to receive a transmit signal from impedance matching circuit 94 via conductors 100, 106 and to provide the transmit signal to feedthrough assembly 90 via conductors 98, 104.

Impedance matching circuit 94 is configured to transfer receive and transmit signals between a first port 100, 106 and a second port 102, 108. The input impedance of the first port 100, 106 may be configured to match the impedance of telemetry antenna 12 and the input impedance of the second port 102, 108 may be configured to match the impedance of telemetry circuit 14. When telemetry system 10 is acting as a receiver, impedance matching circuit 94 is configured to receive a receive signal from protection circuit 92 via conductors 100, 106 and to provide the receive signal to telemetry circuit 14 via conductors 102, 108. When telemetry system 10 is acting as a transmitter, impedance matching circuit 94 is configured to receive a transmit signal from telemetry circuit 14 via conductors 102, 108 and to provide the transmit signal to protection circuit 92 via conductors 100, 106.

Stub filter 18 may be electrically coupled to telemetry conduction path 16 in one of several locations. For example, an end of the conductor in stub filter 18 may be electrically coupled to one of main line conductors 96, 98, 100, 102. When stub filter 18 includes a secondary conductor, an end of the secondary conductor may be electrically coupled to one of secondary line conductors 104, 106, 108. In some examples, secondary line conductors 104, 106, 108 may be electrically coupled to an RF ground. The RF ground may, in some examples, be capacitively coupled to the housing of the device in which implantable telemetry system 10 operates.

Feedthrough assembly 90 separates those components situated outside of the housing of a device in which telemetry system 10 is operating from those situated within the housing. Thus, telemetry antenna 12 is located outside of the housing while protection circuit 92, impedance matching circuit 94 and telemetry circuit 14 are located within the housing. When stub filter is electrically coupled to conductor 96, stub filter 18 may reside completely outside of the housing of the device. When stub filter is electrically coupled to conductors 98, 100, 102, stub filter 18 may reside completely within the device.

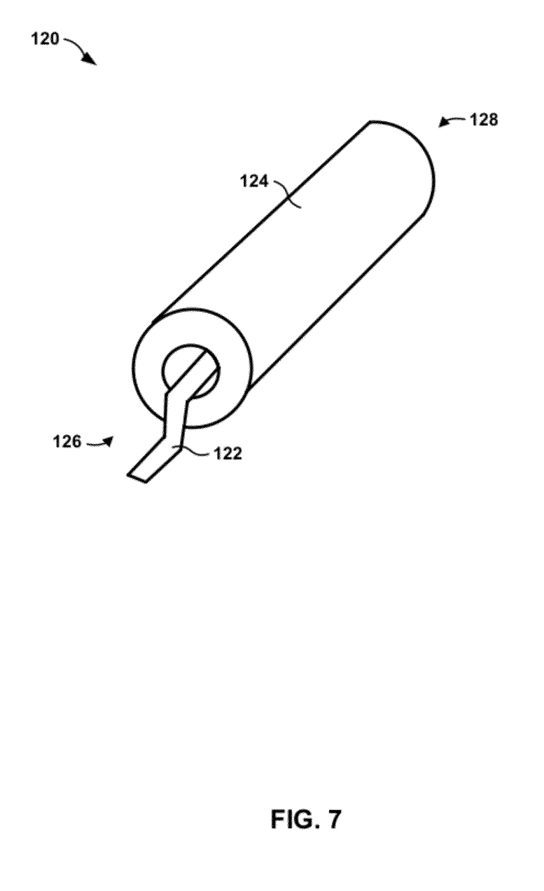

FIG. 7 is a conceptual diagram illustrating an example stub filter 120 that may be utilized in the implantable telemetry system 10 of FIG. 1 according to this disclosure. Stub filter 120 includes a conductor 122 and a dielectric 124.

Conductor 122 includes two ends 126, 128 opposite each other. End 126 of electrode 122 is coupled to telemetry conduction path 16 (FIGS. 1, 2, 5 and 6) at a location thereby forming a junction in telemetry conduction path 16. End 126 may be coupled to telemetry conduction path 16 by soldering, for example, or other electrical connection technique. End 128 is configured in an open circuit configuration. In other words, end 128 is not electrically coupled to another conductor. In some examples, conductor 122 may form an elongated conductor having first end 126 and second end 128. In further examples, conductor 122 may be shaped in substantially a straight line. Conductor 122 may be formed from any conductive material, such as, e.g., copper or silver, gold, etc.

Dielectric 124 is configured to reduce the wavelength of the interfering signal propagating through conductor 122, thereby decreasing the physical length needed to achieve a particular electrical length for stub filter 120. Conductor 122 is disposed within dielectric 124, and in some examples, may surround conductor 122 as shown in FIG. 7.

Conductor 122 has an electrical length approximately equal to one-quarter of the wavelength of the interfering signal when propagating through stub filter 18. Thus, stub filter 120 may form a one-quarter wavelength stub filter. As used herein, the length of stub filter may refer to the length of the transmission medium in the stub filter, e.g., the conductor and/or dielectric within the stub filter. The term electrical length may refer to the length of the transmission medium in the stub filter expressed as a number of wavelengths of the interfering signal when propagating through the transmission medium. In contrast, the term physical length, as used herein, may refer to the length of the transmission medium in the stub filter expressed in units of length independent of the wavelength of the interfering signal. The wavelength of the interfering signal when propagating through the transmission medium may be less than the wavelength of the interfering signal when propagating through free space or air. For example, wavelength of the interfering signal when propagating through the transmission medium may be approximately equal to the product of the free space wavelength of the interfering signal multiplied by the velocity factor for the transmission medium.

During operation, the interfering signal is sent down conductor 122 of stub filter 120. Upon reaching end 128 of the stub filter 120, the interfering signal is reflected back down conductor 122 of stub filter 120 at near 100 percent efficiency. Upon arriving back at end 126, the reflected signal is now approximately 180 degrees out-of-phase of the original signal (because it is now one-half of a wavelength later). The reflected signal adds destructively to the incoming interfering signal, thereby greatly attenuating the interfering signal.

In some examples, stub filter 120 is comprised of a high dielectric material 124 so that the physical length of the stub filter is suitable for use in an implanted medical device or other small geometry applications. The dielectric constant required to obtain a particular physical size suitable for any application can be determined by the following equation:

$$λ = \frac{1}{\sqrt{{μ}_{R} \cdot {ɛ}_{R}}} \cdot \frac{c}{f} \qquad ( 1 )$$

where f is the frequency to be attenuated or cancelled, λ is the wavelength of signal to be attenuated (e.g., the interfering signal) when propagating through a transmission medium, C is the speed of light, μ_{R} is relative permeability of the transmission medium, and ∈_{R} is the dielectric constant value of the transmission medium. In some examples, the frequency to be attenuated or cancelled (f) may be 45 MHz for 1.0 Tesla MRI scanners, 64 MHz for 1.5 Tesla MRI scanners and 128 MHz for 3 Tesla MRI scanners. In equation (1),

$$\frac{1}{\sqrt{{μ}_{R} \cdot {ɛ}_{R}}}$$

may correspond to the velocity factor for the transmission medium, and

$$\frac{c}{f}$$

may correspond to the free space wavelength of the interfering signal having frequency f.

For stub filter 120 to attenuate or cancel a 64 MHz MRI signal, and using a nonferromagnetic material for the transmission medium (thus providing a μ_{R}=1), λ would equal 1.89 inches if an ∈_{R} of 9500 was used for the transmission medium. An ∈_{R} of this magnitude is available, for example, in Z5V Barium Titanate. To design a conductor with an electrical length of one-quarter of the wavelength of the 64 MHz MRI signal (¼ λ), the conductor may have a physical length that is equal to 0.47 inches, which is a suitable length for use in an IMD.

In some examples, conductor 122 may operate as a main conductor with respect to a secondary conductor (not shown) electrically coupled to RF ground. In such examples, the main conductor 122 is in an open circuit configuration with respect to the secondary conductor, i.e., at end 128, main conductor 122 is not electrically coupled to the secondary conductor. In such examples, main conductor 122 and the secondary conductor may form an “open circuit terminated transmission line.” The secondary conductor may be, for example, the secondary conductive path in telemetry conduction path 16. In additional examples, the secondary conductor may be an RF ground plane on a hybrid circuit board within an implantable medical device in which stub filter 120 operates.

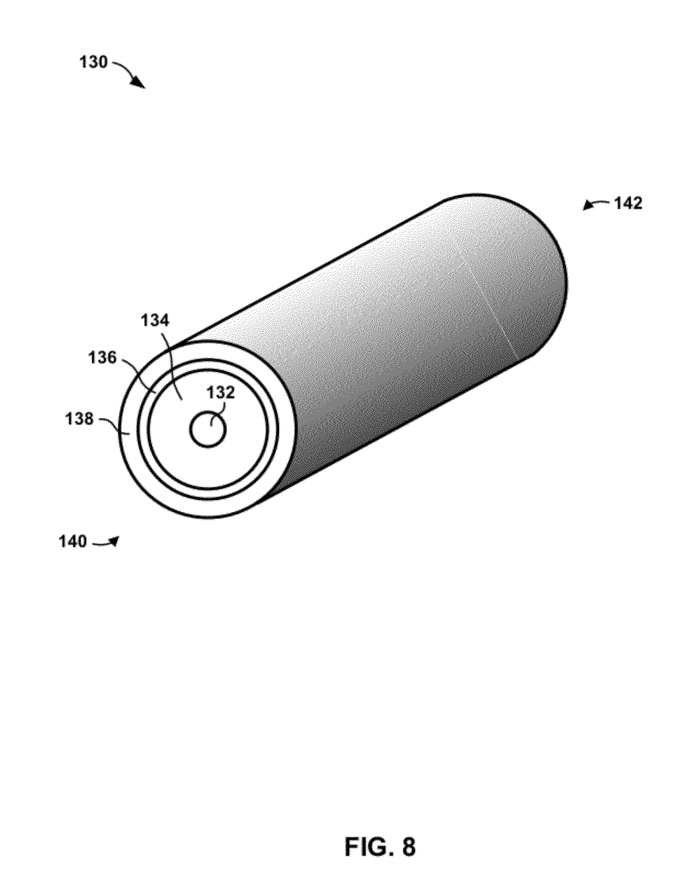

FIG. 8 is a conceptual diagram illustrating another example stub filter 130 that may be used in the implantable telemetry system 10 of FIG. 1 according to this disclosure. Stub filter 130 includes a main conductor 132, a dielectric 134, a secondary conductor 136, and an insulative shield 138. Main conductor 132 and dielectric 134 may correspond to conductor 122 and dielectric 124, and will not be described in further detail.

Secondary conductor 136 surrounds main conductor 132 and dielectric 134, and insulative shield 138 surrounds secondary conductor 136. Dielectric 134 is configured to electrically isolate main conductor 132 and secondary conductor 136. Dielectric 134 is also configured to reduce the wavelength of the interfering signal propagating through conductors 132, 136, thereby decreasing the physical length needed to achieve a particular electrical length for stub filter 130.

Main conductor 132 may be electrically coupled at end 140 to a main line conductive path in telemetry conduction path 16 (FIGS. 1, 2, 5 and 6). Secondary conductor 136 may be electrically coupled at end 140 to a secondary conductive path within telemetry conduction path 16. Ends 142 of main conductor 132 and secondary conductor 136 are configured in an open circuit configuration. Thus, stub filter 130 may be referred to as an open circuit terminated transmission line.

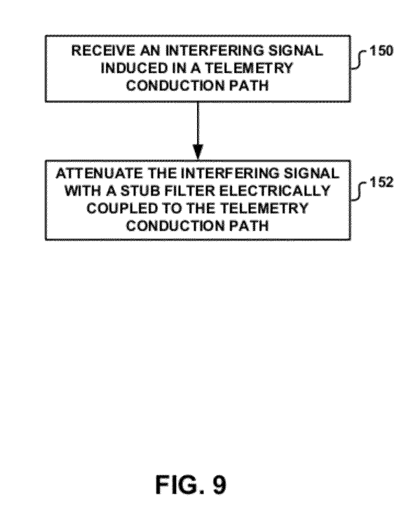

FIG. 9 is a flow diagram illustrating an example technique for attenuating an interfering signal within an implantable telemetry system according to this disclosure. The technique illustrated in FIG. 9 may be implemented, for example, with any of the implantable telemetry systems and stub filters described above with respect to FIGS. 1, 2 and 5-8.

Stub filter 18 receives an interfering signal induced in a telemetry conduction path 16 (150). Stub filter 18 attenuates the interfering signal (152). Stub filter 18 is electrically coupled to the telemetry conduction path 16 (152). The telemetry conduction path 16 may include a first end 20 electrically coupled to a telemetry antenna 12 and a second end 22 electrically coupled to a telemetry circuit 14 disposed within a housing of the implantable medical device. The stub filter 18 used for attenuating the interfering signal may include a dielectric and an conductor disposed within the dielectric. The conductor may include a first end electrically coupled to the telemetry conduction path and a second end configured in an open circuit configuration. The conductor may have an electrical length approximately equal to one-quarter of the wavelength of the interfering signal when propagating through the stub filter 18.

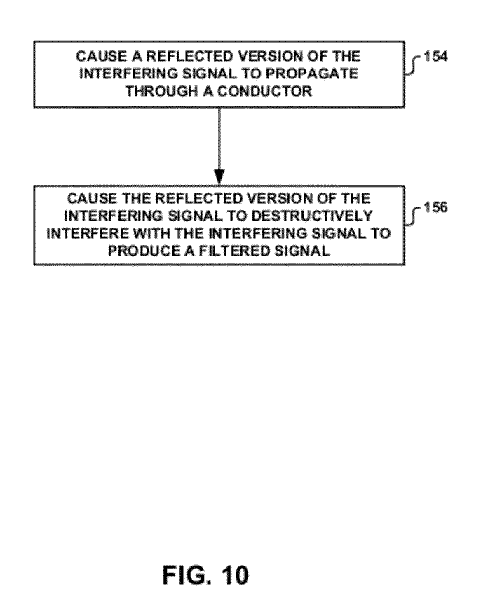

FIG. 10 is a flow diagram illustrating another example technique for attenuating an interfering signal within an implantable telemetry system according to this disclosure. The technique illustrated in FIG. 10 may be implemented, for example, with any of the implantable telemetry systems and stub filters described above with respect to FIGS. 1, 2 and 5-8. The technique illustrated in FIG. 10 may, in some cases, correspond to process box 152 in FIG. 9.

Stub filter 18 causes a reflected version of the interfering signal to propagate through a conductor (154). The reflected version of the interfering signal may propagate through the conductor in a direction opposite the interfering signal (i.e., the incident wave). At the open circuit end of stub filter 18, the reflected version of the interfering signal may be substantially in-phase with the incident wave. At the junction where telemetry conduction path 16 and stub filter 18 meet, the reflected version of the interfering signal may be approximately 180 degrees out-of-phase with the incident wave. Stub filter 18 causes the reflected version of the interfering signal to destructively interfere with the interfering signal to produce a filtered signal (156). The destructive interference may occur at the junction where telemetry conduction path 16 and stub filter 18 meet. The frequency components attributable to the interfering signal may be substantially attenuated and/or eliminated in the filtered signal. In some examples, the filtered signal may be a standing wave of substantially zero magnitude.

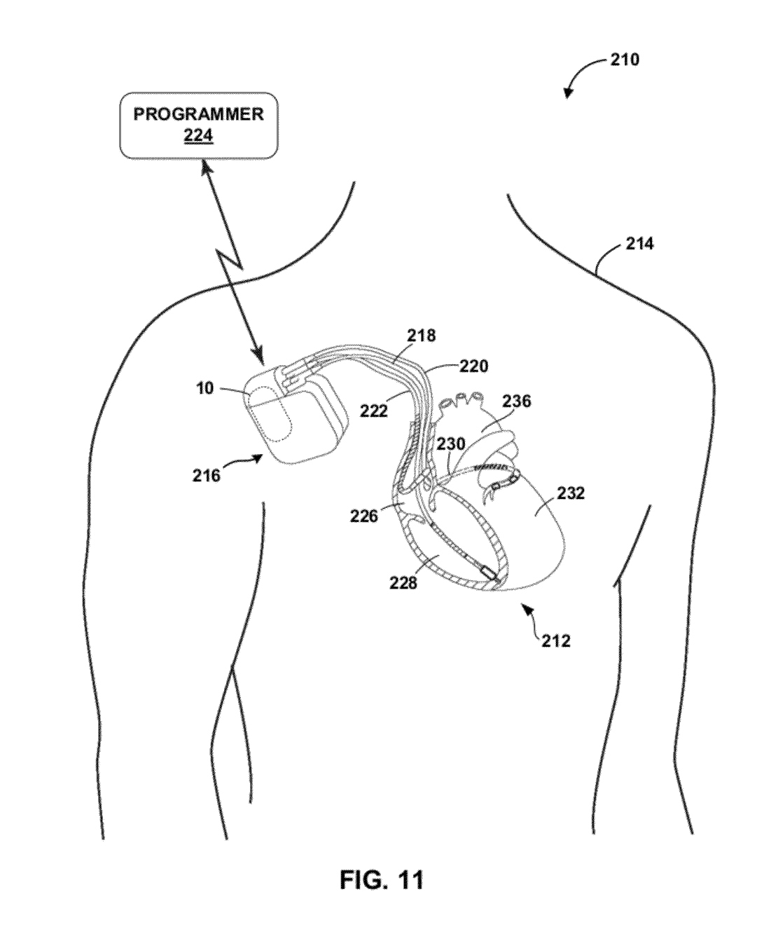

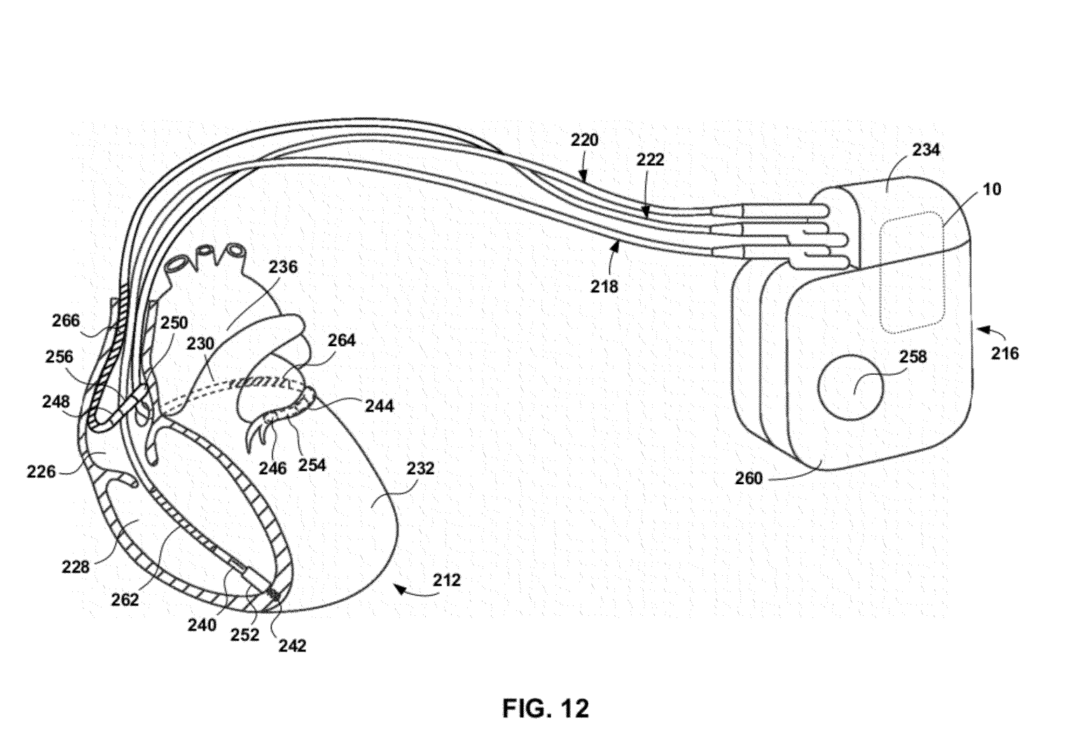

FIG. 11 is a conceptual diagram illustrating an example therapy system 210 that may utilize the implantable telemetry system 10 of FIG. 1 according to this disclosure. Therapy system 210 is configured to provide therapy to heart 212 of patient 214. Patient 214 is ordinarily, but not necessarily, a human patient. Therapy system 210 includes IMD 216, leads 218, 220, 222, and programmer 224. IMD 216 is coupled to each of leads 218, 220, 222.

IMD 216 may be, for example, a device that provides cardiac rhythm management therapy to heart 212, and may include, for example, an implantable pacemaker, cardioverter, and/or defibrillator that provides therapy to heart 212 of patient 214 via electrodes coupled to one or more of leads 218, 220, and 222. In some examples, IMD 216 may deliver pacing pulses, but not cardioversion or defibrillation shocks, while in other examples, IMD 216 may deliver cardioversion and/or defibrillation shocks in addition to pacing pulses. In additional examples, IMD 216 may provide cardiac resynchronization therapy in addition to or in lieu of pacing pulses, cardioversion shocks, and/or defibrillation shocks.

Leads 218, 220, 222 extend into the heart 212 of patient 214 to sense electrical activity of heart 212 and/or deliver electrical stimulation to heart 212. In the example shown in FIG. 11, right ventricular (RV) lead 218 extends through one or more veins (not shown), the superior vena cava (not shown), and right atrium 226, and into right ventricle 228. Left ventricular (LV) coronary sinus lead 220 extends through one or more veins, the vena cava, right atrium 226, and into the coronary sinus 230 to a region adjacent to the free wall of left ventricle 232 of heart 212. Right atrial (RA) lead 222 extends through one or more veins and the vena cava, and into right atrium 226 of heart 212. In other examples, therapy system 210 may include an additional lead or lead segment (not shown in FIG. 11) that deploys one or more electrodes within the vena cava or other vein. These electrodes may allow alternative electrical sensing configurations that may provide improved sensing accuracy in some patients.

IMD 216 senses electrical signals attendant to the depolarization and repolarization of heart 212 via electrodes coupled to at least one of the leads 218, 220, 222. In some examples, IMD 216 provides pacing pulses to heart 212 based on the electrical signals sensed within heart 212. These electrical signals sensed within heart 212 may also be referred to as cardiac signals or electrical cardiac signals. The configurations of electrodes used by IMD 216 for sensing and pacing may be unipolar or bipolar. IMD 216 may also provide defibrillation therapy and/or cardioversion therapy via electrodes located on at least one of the leads 218, 220, 222. IMD 216 may detect arrhythmia of heart 212, such as fibrillation of ventricles 228 and 232, and deliver cardioversion or defibrillation therapy to heart 212 in the form of electrical pulses. In some examples, IMD 216 may be programmed to deliver a progression of therapies, e.g., pulses with increasing energy levels, until a tachyarrhythmia of heart 212 is stopped. IMD 216 detects tachycardia or fibrillation employing one or more tachycardia or fibrillation detection techniques known in the art.

In some examples, programmer 224 may be a handheld computing device, computer workstation, or networked computing device. Programmer 224 includes a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 224 can additionally or alternatively include a peripheral pointing device, such as a mouse, via which a user may interact with the user interface. In some examples, a display of programmer 224 may include a touch screen display, and a user may interact with programmer 224 via the display. It should be noted that the user may also interact with programmer 224 or IMD 216 remotely via a networked computing device.

A user, such as a physician, technician, surgeon, electrophysiologist, or other clinician, may interact with programmer 224 to communicate with IMD 216. For example, the user may interact with programmer 224 to retrieve physiological or diagnostic information from IMD 216. A user may also interact with programmer 224 to program IMD 216, e.g., select values for operational parameters of IMD 216.

For example, the user may use programmer 224 to retrieve information from IMD 216 regarding the rhythm of heart 212, trends therein over time, or tachyarrhythmia episodes. As another example, the user may use programmer 224 to retrieve information from IMD 216 regarding other sensed physiological parameters of patient 214, such as electrical depolarization/repolarization signals from the heart (referred to as “electrogram” or EGM), intracardiac or intravascular pressure, activity, posture, respiration, heart rate, heart sounds, or thoracic impedance. As another example, the user may use programmer 224 to retrieve information from IMD 216 regarding the performance or integrity of IMD 216 or other components of system 210, such as leads 218, 220 and 222, or a power source of IMD 216.

The user may use programmer 224 to program a therapy progression, select electrodes used to deliver defibrillation shocks, select waveforms for the defibrillation shocks, or select or configure a fibrillation detection algorithm for IMD 216. The user may also use programmer 224 to program similar aspects of other therapies provided by IMD 216, such as cardioversion or pacing therapies. In some examples, the user may activate certain features of IMD 216 by entering a single command via programmer 224, such as depression of a single key or combination of keys of a keypad or a single point-and-select action with a pointing device.

According to this disclosure IMD 216 includes an implantable telemetry system 10. IMD 216 and programmer 224 may communicate via wireless communication using implantable telemetry system 10. Implantable telemetry system 10 uses radio frequency (RF) telemetry techniques, but other techniques are also contemplated. In some examples, programmer 224 may include a programming head that may be placed proximate to the patient's body near the IMD 216 implant site in order to improve the quality or security of communication between IMD 216 and programmer 224.

Implantable telemetry system 10 of IMD 216 may have one or more components configured such that, when patient 214 and/or IMD 216 is in the presence of a source of electromagnetic interference, such as, e.g., interference generated by an MRI scan, some or all of the components within IMD 216 are protected from some or all of the effects of such interference. For example, IMD 216 may include a telemetry conduction path comprising a first end electrically coupled to a telemetry antenna and a second end electrically coupled to a telemetry circuit disposed within a housing of IMD 216. IMD 216 may further include a stub filter electrically coupled to the telemetry conduction path and configured to attenuate an interfering signal induced in the telemetry conduction path. The stub filter may include a dielectric and a conductor disposed within the dielectric. The conductor may include a first end electrically coupled to the telemetry conduction path and a second end configured in an open circuit configuration. The conductor may have an electrical length approximately equal to one-quarter of the wavelength of the interfering signal when propagating through the stub filter.

FIG. 12 is a conceptual diagram illustrating IMD 216 and leads 218, 220 and 222 of therapy system 210 in greater detail. As shown in FIG. 12, IMD 216 includes a housing 260 and a connector block 234. Leads 218, 220, 222 may be electrically coupled to a signal generator and a sensing module of IMD 216 via connector block 234. In some examples, proximal ends of leads 218, 220, 222 may include electrical contacts that electrically couple to respective electrical contacts within connector block 234 of IMD 216. In addition, in some examples, leads 218, 220, 222 may be mechanically coupled to connector block 234 with the aid of set screws, connection pins, snap connectors, or another suitable mechanical coupling mechanism.

Each of the leads 218, 220, 222 includes an elongated insulative lead body, which may carry a number of concentric coiled conductors separated from one another by tubular insulative sheaths. Other lead configurations are also contemplated, such as configurations that do not include coiled conductors. In the illustrated example, bipolar electrodes 240 and 242 are located proximate to a distal end of lead 218 in RV 228. In addition, bipolar electrodes 244 and 246 are located proximate to a distal end of lead 220 in LV 232 and bipolar electrodes 248 and 250 are located proximate to a distal end of lead 222 in RA 226. Although no electrodes are located in LA 236 in the illustrated example, other examples may include electrodes in LA 236.

Electrodes 240, 244, and 248 may take the form of ring electrodes, and electrodes 242, 246, and 250 may take the form of extendable helix tip electrodes mounted retractably within insulative electrode heads 252, 254, and 256, respectively. In other examples, one or more of electrodes 242, 246, and 250 may take the form of small circular electrodes at the tip of a tined lead or other fixation element. Leads 218, 220, 222 also include elongated electrodes 262, 264, 266, respectively, which may take the form of a coil. Each of the electrodes 240, 242, 244, 246, 248, 250, 262, 264, and 266 may be electrically coupled to a respective one of the conductors within the lead body of its associated lead 218, 220, 222, and thereby coupled to respective ones of the electrical contacts on the proximal end of leads 218, 220, 222.

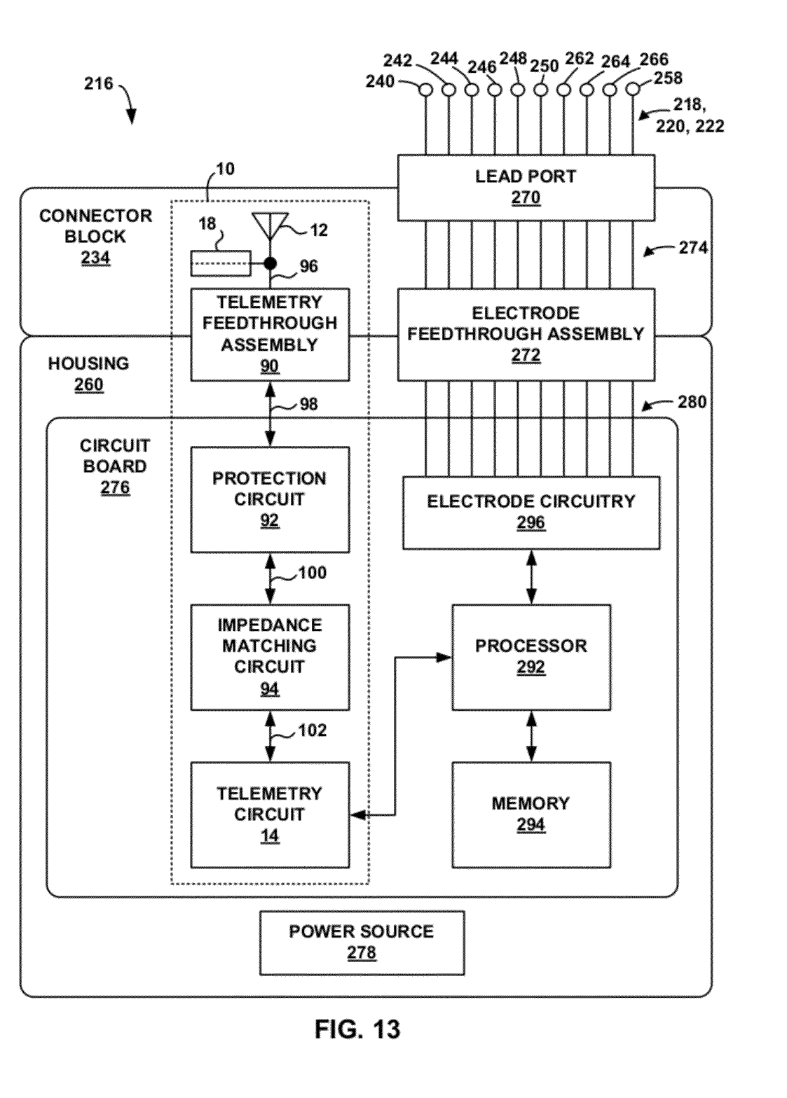

In some examples, as illustrated in FIG. 12, IMD 216 includes one or more housing electrodes, such as housing electrode 258, which may be formed integrally with an outer surface of hermetically-sealed housing 260 of IMD 216 or otherwise coupled to housing 260. Housing electrode 258 may be defined, in some examples, by an uninsulated portion of an outward facing portion of housing 260 of IMD 216. Other divisions between insulated and uninsulated portions of housing 260 may be employed to define two or more housing electrodes. In some examples, housing electrode 258 comprises substantially all of housing 260. As described in further detail with reference to FIG. 13, housing 260 may enclose a signal generator that generates therapeutic stimulation, such as cardiac pacing pulses and defibrillation shocks, as well as a sensing module for monitoring the rhythm of heart 212.

IMD 216 may sense electrical signals attendant to the depolarization and repolarization of heart 212 via electrodes 240, 242, 244, 246, 248, 250, 258, 262, 264, and 266. The electrical signals are conducted to IMD 216 from the electrodes via the respective leads 218, 220, 222 or, in the case of housing electrode 258, a conductor couple to housing electrode 258. IMD 216 may sense such electrical signals via any bipolar combination of electrodes 240, 242, 244, 246, 248, 250, 258, 262, 264, and 266. Furthermore, any of the electrodes 240, 242, 244, 246, 248, 250, 258, 262, 264, and 266 may be used for unipolar sensing in combination with housing electrode 258.

Any multipolar combination of two or more of electrodes 240, 242, 244, 246, 248, 250, 258, 262, 264, and 266 may be considered a sensing electrode configuration. Usually, but not necessarily, a sensing electrode configuration is a bipolar electrode combination on the same lead, such as electrodes 240 and 242 of lead 218. On one lead having three electrodes, there may be at least three different sensing electrode configurations available to IMD 216. These sensing electrode configurations are, for the example of lead 218, tip electrode 242 and ring electrode 240, tip electrode 242 and elongated electrode 262, and ring electrode 240 and elongated electrode 262. However, some examples may utilize sensing electrode configurations having electrodes of two different leads. Further, a sensing electrode configuration may utilize housing electrode 258, which may provide a unipolar sensing electrode configuration. In some examples, a sensing electrode configuration may comprise multiple housing electrodes 258. In any sensing electrode configuration, the polarity of each electrode in the may be configured as appropriate for the application of the sensing electrode configuration.

In some examples, IMD 216 delivers pacing pulses via bipolar combinations of electrodes 240, 242, 244, 246, 248 and 250 to produce depolarization of cardiac tissue of heart 212. In additional examples, IMD 216 delivers pacing pulses via any of electrodes 240, 242, 244, 246, 248 and 250 in combination with housing electrode 258 in a unipolar configuration. Furthermore, IMD 216 may deliver cardioversion or defibrillation shocks to heart 212 via any combination of elongated electrodes 262, 264, 266, and housing electrode 258. Electrodes 258, 262, 264, 266 may also be used to deliver cardioversion shocks to heart 212. Electrodes 262, 264, 266 may be fabricated from any suitable electrically conductive material, such as, but not limited to, platinum, platinum alloy, Titanium nitride or other materials known to be usable in implantable defibrillation electrodes.

The configuration of therapy system 210 illustrated in FIGS. 11 and 12 is merely one example of a therapy system in which the techniques in this disclosure may be applied. In other examples, a therapy system may include epicardial leads and/or patch electrodes instead of or in addition to the implanted leads 218, 220, 222 illustrated in FIG. 11. Further, housing 260 of IMD 216 need not be implanted within patient 214. In examples in which housing 260 is not implanted in patient 214, IMD 216 may deliver defibrillation pulses and other therapies to heart 212 via percutaneous leads that extend through the skin of patient 214 to a variety of positions within or outside of heart 212.

In other examples of therapy systems that provide electrical stimulation therapy to heart 212, a therapy system may include any suitable number of leads coupled to IMD 216, and each of the leads may extend to any location within or proximate to heart 212. For example, a therapy system may include a single chamber or dual chamber device rather than a three-chamber device as shown in FIG. 11. In a single chamber configuration, IMD 216 is electrically connected to a single lead 220 that includes stimulation and sense electrodes within LV 232. In one example of a dual chamber configuration, IMD 216 is electrically connected to a single lead that includes stimulation and sense electrodes within LV 232 as well as sense and/or stimulation electrodes within RA 226. In another example of a dual chamber configuration, IMD 216 is connected to two leads that extend into a respective one of the RA 228 and LV 232. Other lead configurations are contemplated, and the techniques in this disclosure are not limited to any particular number of leads or configuration of leads.

The techniques of this disclosure may be used in an IMD that provides other types of electrical stimulation therapy other than cardiac rhythm management therapy or in devices that provide no therapy at all, but only monitor a condition of a patient. For example, the IMD may be a device that provides electrical stimulation to a tissue site of patient 212 proximate a muscle, organ or nerve, such as a tissue proximate a vagus nerve, spinal cord, brain, stomach, pelvic floor or the like. Moreover, the techniques may be used to operate an IMD that provides other types of therapy, such as drug delivery or infusion therapies. In addition, the techniques in this disclosure may be used in monitoring devices that sense physiological parameters of a patient, but do not provide therapy to the patient. As such, description of these techniques in the context of cardiac rhythm management therapy should not be limiting of the techniques as broadly described in this disclosure.

FIG. 13 is a block diagram illustrating an example configuration of IMD 216. As shown in FIG. 13, IMD 216 includes a connector block 234 and a housing 260. IMD 216 also includes implantable telemetry system 10 which is formed within both of connector block 234 and housing 236. Implantable telemetry system 10 is substantially similar to the implantable telemetry system described above with respect to FIG. 6 except that the secondary conductors are not shown. Identically numbered components between FIGS. 6 and 13 may have the same or similar functionality and be constructed from the same or similar components. Thus, in the interest of brevity and to avoid redundancy, these similar numbered components will not be described in further detail.

Implantable telemetry system 10 includes a main line conductive path formed by telemetry feedthrough assembly 90, protection circuit 92, impedance matching circuit 94 and main line conductors 96, 98, 100, 102. As shown in FIG. 13, a one-quarter wavelength (at 45 MHz, 64 MHz or 128 MHz for MRI RF frequencies) stub filter 18 is electrically coupled to the main line conductive path at conductor 96 thereby forming a junction as discussed above with respect to FIG. 2. The junction is located in connector block 234 between antenna 12 and telemetry feedthrough assembly 90. Stub filter 18 may create a reflected signal that attenuates an interfering signal induced by RF interference from an MRI scanner (or other device generating an interfering radiating signal). The attenuation may prevent the interfering signal from reaching various components on circuit board 276, such as, e.g., telemetry circuit 14, processor 292 and/or electrode circuitry 296. In this manner, some or all of the circuitry within housing 260 may be protected from interference caused by such an interfering signal.

Although stub filter 18 is illustrated in the example of FIG. 13 as being electrically coupled to the main line conductive path at main line conductor 96, in additional examples, stub filter 18 may be electrically coupled to main line conductive path at any of main line conductors 98, 100, 102. In additional examples, stub filter 18 may be electrically coupled to the main line conductive path within one of telemetry feedthrough assembly 90, protection circuit 92 and impedance matching circuit 94.

As shown in FIG. 13, stub filter 18 is completely contained within connector block 234 of IMD 216 and external to housing 276 of IMD 216. However, in examples where stub filter 18 is electrically coupled to the main line conductive path on the opposite of telemetry feedthrough assembly 90, stub filter 18 may be completely contained within housing 260 of IMD 216. In additional examples, telemetry antenna 12 may be disposed outside of both housing 260 and connector block 234. In such examples, stub filter 18 may, in some examples, be electrically coupled to the main line conductive path outside of both housing 260 and connector block 234.

Connector block 234 is configured to provide a region external to housing 60 in which a telemetry antenna may be disposed. Connector block 234 is further configured to couple leads 218, 220, 222 to IMD 216. Connector block 234 includes lead port 270, electrode feedthrough assembly 272 and conductors 274.

Lead port 270 may be configured to secure leads 218, 220, 222 to connector block 234. Each of leads 218, 220, 222 may have a lead connector on the proximal end of the lead, which can be inserted into lead port 270. The proximal lead connector may include electrical contacts, each of which may be coupled to a respective electrode at the distal end of the lead via a respective lead conductor. Lead port 270 may include a suitable means for locking the lead connector into lead port 270 such that, when inserted, each of the electrical contacts is electrically coupled to a respective conductor 274. For example, lead port 270 may include one or more lead receptacles each of which are configured to receive and lock one of leads 218, 220, 222 in place.

Electrode feedthrough assembly 272 is configured to provide electrical communication between the outside and inside of hermetically-sealed housing 260. Electrode feedthrough assembly may, in some examples, be affixed to a side-wall of housing 260. Electrode feedthrough assembly 272 may include a plurality of feedthrough conductors, each of which may have a first terminal that is disposed outside of housing 260 and a second terminal that is disposed inside of housing 260. The first terminal of each feedthrough conductor may be electrically coupled to a respective conductor 274, which is in turn electrically coupled to a respective lead 218, 220, 222. A second terminal of each feedthrough conductor may be electrically coupled to electrode circuitry 296 on circuit board via a respective conductor 280. In some examples, the feedthrough conductors may include one or more feedthrough pins.

Electrode feedthrough assembly 272 may, in some examples, include one or more feedthrough capacitors. For each feedthrough capacitor, a first terminal may be electrically coupled to a portion of a respective feedthrough conductor (e.g., the feedthrough pin) and a second terminal may be electrically coupled to housing 260. In addition, the first terminal of the feedthrough capacitor may be electrically coupled to a respective contact of an implantable lead, e.g., via a respective conductor 274. The feedthrough capacitors may be configured to block or attenuate electromagnetic interference (EMI) from entering housing 260 and electronic circuitry of IMD 216. In some examples, the feedthrough capacitors may attenuate any incoming high frequency electrical energy, e.g., any frequency of electrical energy that is above approximately 3 MHz. Although the feedthrough capacitors are described as being included within electrode feedthrough assembly 272, in other examples, the feedthrough capacitors may be completely or partially outside of electrode feedthrough assembly 272.

Conductors 274 are configured to provide electrical communication between lead port 270 and electrode feedthrough assembly 272. Conductors 274 may be implemented as a conductive wire or as an interconnect ribbon. Although ten conductors are illustrated as passing through electrode feedthrough assembly 272, the number of conductors that pass through electrode feedthrough assembly 272 typically depends on the number of electrodes and the number of leads of IMD 216 and may be more or less than ten.

Telemetry feedthrough assembly 90 is configured to transfer receive and transmit signals between the outside and inside of housing 60. Telemetry feedthrough assembly 90 may include a conductor having a first end electrically coupled to main line conductor 96 and a second end electrically coupled to main line conductor 98. Unlike electrode feedthrough assembly 272, telemetry feedthrough assembly 90 may, in some examples, not include a feedthrough capacitor. The exclusion of a feedthrough capacitor in such examples may prevent legitimate telemetry signals from being shunted to ground.

Although telemetry feedthrough assembly 90 is illustrated in FIG. 13 as a separate assembly from that of electrode feedthrough assembly 272, in other examples, telemetry feedthrough assembly 90 and electrode feedthrough assembly 272 may be combined into a single feedthrough assembly. Moreover, although IMD 216 in FIG. 13 only includes a single conductor (e.g., a single feedthrough pin) passing through telemetry feedthrough assembly 90, other examples may include more than one conductor or feedthrough pin.

Housing 260 may be configured to shield the electrical components contained inside of housing 260 from body fluids of patient 214 and from electromagnetic interference. As illustrated in FIG. 13, housing 260 includes telemetry feedthrough assembly 90 electrode feedthrough assembly 272, circuit board 276 and power source 278.

Circuit board 276 may be a substrate that is used to mechanically support and electrically connect electrical components contained on circuit board 276. In some examples, circuit board 276 may be a printed circuit board (PCB) or a printed wiring board (PWB). Circuit board 276 may be electrically coupled to electrode feedthrough assembly 272 via conductors 280 (e.g., ribbon bonds). Circuit board 276 may also be electrically coupled to telemetry feedthrough assembly via conductor 98. In addition, circuit board 276 may be electrically coupled to power source 278. Circuit board 276 includes telemetry circuit 14, protection circuit 92, impedance matching circuit 94, processor 292, memory 294 and electrode circuitry 296.

Conductors 280 are configured to provide electrical communication between electrode feedthrough assembly 272 and circuit board 276. Each of conductors 280 may have a first end or terminal that is electrically coupled to a respective feedthrough conductor within electrode feedthrough assembly 272, and a second end or terminal that is electrically coupled to a respective pad 282 on printed circuit board 276. In some examples, one or more of the ribbon bonds 280 may include a laser ribbon bond that is laser-welded to a feedthrough pin at a first end and to a contact pad 282 at a second end.

Processor 292 may include any one or more of a microprocessor, a controller, a digital signal processor (DSP), an application specific integrated circuit (ASIC), a field-programmable gate array (FPGA), or equivalent discrete or integrated logic circuitry. In some examples, processor 292 may include multiple components, such as any combination of one or more microprocessors, one or more controllers, one or more DSPs, one or more ASICs, or one or more FPGAs, as well as other discrete or integrated logic circuitry. The functions attributed to processor 292 herein may be embodied as software, firmware, hardware or any combination thereof.

Processor 292 controls a signal generator in electrode circuitry 296 to deliver stimulation therapy to heart 212. Processor 292 may control the signal generator to deliver stimulation according to a selected one or more therapy programs, which may be stored in memory 294. For example, processor 292 may control the signal generator to deliver electrical pulses with the amplitudes, pulse widths, frequencies, or electrode polarities specified by the selected one or more therapy programs.

Memory 294 may include computer-readable instructions that, when executed by processor 292, cause IMD 216 and processor 292 to perform various functions attributed to IMD 216 and processor 292 herein. Memory 294 may include any volatile, non-volatile, magnetic, optical, or electrical media, such as a random access memory (RAM), read-only memory (ROM), non-volatile RAM (NVRAM), static RAM (SRAM), electrically-erasable programmable ROM (EEPROM), flash memory, or any other digital media.

Electrode circuitry 296 may include a signal generator that is configured to generate and deliver electrical stimulation therapy to heart 212. The signal generator may deliver pacing pulses via ring electrodes 240, 244, 248 coupled to leads 218, 220, and 222, respectively, and/or helical electrodes 242, 246, and 250 of leads 218, 220, and 222, respectively. For example, the signal generator may deliver a pacing stimulus to LV 232 (FIG. 12) of heart 212 via at least two electrodes 244, 246 (FIG. 12). As another example, the signal generator may deliver defibrillation shocks to heart 212 via at least two electrodes 258, 262, 264, 266. In some examples, the signal generator delivers pacing, cardioversion, or defibrillation stimulation in the form of electrical pulses. In other examples, the signal generator may deliver one or more of these types of stimulation in the form of other signals, such as sine waves, square waves, or other substantially continuous time signals.

In some examples, the signal generator may include a switch module and processor 292 may use the switch module to select, e.g., via a data/address bus, which of the available electrodes are used to deliver pacing pulses, cardioversion shocks, or defibrillation shocks. The switch module may include a switch array, switch matrix, multiplexer, or any other type of switching device suitable to selectively couple stimulation energy to selected electrodes. In other examples, however, the signal generator may independently deliver stimulation to electrodes 240, 242, 244, 246, 248, 250, 258, 262, 264, 266 or selectively sense via one or more of electrodes 240, 242, 244, 246, 248, 250, 258, 262, 264, 266 without a switch matrix.

Electrode circuitry 296 may further include a sensing module that is configured to monitor signals from at least one of electrodes 240, 242, 244, 246, 248, 250, 258, 262, 264, 266 in order to monitor electrical activity of heart 212. For example, the sensing module may sense atrial events (e.g., a P-wave) with electrodes 248, 250, 266 within RA 226 or sense an LV 232 event (e.g., an R-wave) with electrodes 244, 246, 264 within LV 232. The sensing module may also include a switch module to select which of the available electrodes are used to sense the heart activity. In some examples, processor 292 may select the electrodes that function as sense electrodes, or the sensing electrode configuration, via the switch module within the electrical sensing module, e.g., by providing signals via a data/address bus. In some examples, the sensing module may include multiple sensing channels, each of which may comprise an amplifier. In response to the signals from processor 292, the switch module of within the sensing module may couple the outputs from the selected electrodes to one or more of the sensing channels.

In some examples, the sensing module may include a plurality of channels. One channel of the sensing module may include an R-wave amplifier that receives signals from electrodes 240 and 242, which are used for pacing and sensing in RV 228 of heart 212. Another channel may include another R-wave amplifier that receives signals from electrodes 244 and 246, which are used for pacing and sensing proximate to LV 232 of heart 212. In some examples, in one operating mode of the sensing module, the R-wave amplifiers may take the form of an automatic gain controlled amplifier that provides an adjustable sensing threshold as a function of the measured R-wave amplitude of the heart rhythm.

In addition, in some examples, one channel of the sensing module may include a P-wave amplifier that receives signals from electrodes 248 and 250, which are used for pacing and sensing in right atrium 226 of heart 212. In some examples, in one operating mode of the sensing module, the P-wave amplifier may take the form of an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of the sensing module may be selectively coupled to housing electrode 258, or elongated electrodes 262, 264, or 266, with or instead of one or more of electrodes 240, 242, 244, 246, 248 or 250, e.g., for unipolar sensing of R-waves or P-waves in any of chambers 226, 228, or 232 of heart 212.

In some examples, the sensing module may include a channel that comprises an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in memory 294 as an EGM. In some examples, the storage of such EGMs in memory 294 may be under the control of a direct memory access circuit. Processor 292 may employ digital signal analysis techniques to characterize the digitized signals stored in memory 294 to detect and classify the patient's heart rhythm from the electrical signals. Processor 292 may detect and classify the heart rhythm of patient 214 by employing any of the numerous signal processing methodologies known in the art.

In some examples, processor 292 may also include programmable counters which control the basic time intervals associated with DDD, VVI, DVI, VDD, AAI, DDI, DDDR, VVIR, DVIR, VDDR, AAIR, DDIR and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

In some examples, processor 292 may include atrial and ventricular pacing escape intervals, refractory periods during which sensed P-waves and R-waves are ineffective to restart timing of the escape intervals, and the pulse widths of the pacing pulses. As another example, processor 292 may define a blanking period, and provide signals from the sensing module to blank one or more channels, e.g., amplifiers, for a period during and after delivery of electrical stimulation to heart 212. The durations of these intervals may be determined by processor 292 in response to stored data in memory 294. Processor 292 may also determine the amplitude of the cardiac pacing pulses.

During pacing, escape interval counters within processor 292 may be reset upon sensing of R-waves and P-waves with detection channels of the electrical sensing module. The signal generator may include pacer output circuits that are coupled, e.g., selectively by a switching module, to any combination of electrodes 240, 242, 244, 246, 248, 250, 258, 262, or 266 appropriate for delivery of a bipolar or unipolar pacing pulse to one of the chambers of heart 212. Processor 292 may reset the escape interval counters upon the generation of pacing pulses by the signal generator, and thereby control the basic timing of cardiac pacing functions, including anti-tachyarrhythmia pacing.

The value of the count present in the escape interval counters when reset by sensed R-waves and P-waves may be used by processor 292 to measure the durations of R-R intervals, P-P intervals, P-R intervals and R-P intervals, which are measurements that may be stored in memory 294. Processor 292 may use the count in the interval counters to detect a tachyarrhythmia event, such as ventricular fibrillation event or ventricular tachycardia event. Upon detecting a threshold number of tachyarrhythmia events, processor 292 may identify the presence of a tachyarrhythmia episode, such as a ventricular fibrillation episode, a ventricular tachycardia episode, or a non-sustained tachycardia (NST) episode. Examples of tachyarrhythmia episodes that may qualify for delivery of responsive therapy include a ventricular fibrillation episode or a ventricular tachyarrhythmia episode.

In some examples, processor 292 may operate as an interrupt driven device that is responsive to interrupts from pacer timing and control module 292, where the interrupts may correspond to the occurrences of sensed P-waves and R-waves and the generation of cardiac pacing pulses. Any necessary mathematical calculations to be performed by processor 292 and any updating of the values or intervals controlled by processor 292 may take place following such interrupts. A portion of memory 294 may be configured as a plurality of recirculating buffers, capable of holding a series of measured intervals, which may be analyzed by processor 292 in response to the occurrence of a pace or sense interrupt to determine whether the patient's heart 212 is presently exhibiting atrial or ventricular tachyarrhythmia.

In some examples, an arrhythmia detection method may include any suitable tachyarrhythmia detection algorithms. In one example, processor 292 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998, or in U.S. Pat. No. 7,930,024 to Kevin T. Ousdigian, entitled, “REDUCING INAPPROPRIATE DELIVERY OF THERAPY FOR SUSPECTED NON-LETHAL ARRHYTHMIAS,” which issued on Apr. 19, 2011. U.S. Pat. No. 5,545,186 to Olson et al., U.S. Pat. No. 5,755,736 to Gillberg et al., and U.S. Pat. No. 7,930,024 to Kevin T. Ousdigian are incorporated herein by reference in their entireties. However, other arrhythmia detection methodologies may also be employed by processor 292 in other examples.

In additional examples, processor 292 may identify the presence of an atrial or ventricular tachyarrhythmia episode by detecting a series of tachyarrhythmia events (e.g., R-R or P-P intervals having a duration less than or equal to a threshold) of an average rate indicative of tachyarrhythmia or an unbroken series of short R-R or P-P intervals. The thresholds for determining the R-R or P-P interval that indicates a tachyarrhythmia event may be stored within memory 294 of IMD 216. In addition, the number of tachyarrhythmia events that are detected to confirm the presence of a tachyarrhythmia episode may be stored as a number of intervals to detect (NID) threshold value in memory 294. In some examples, processor 292 may also identify the presence of the tachyarrhythmia episode by detecting a variable coupling interval between the R-waves of the heart signal. For example, if the interval between successive tachyarrhythmia events varies by a particular percentage or the differences between the coupling intervals are higher than a given threshold over a predetermined number of successive cycles, processor 292 may determine that the tachyarrhythmia is present.

In the event that processor 292 detects an atrial or ventricular tachyarrhythmia based on signals from the electrical sensing module, and an anti-tachyarrhythmia pacing regimen is desired, timing intervals for controlling the generation of anti-tachyarrhythmia pacing therapies by the signal generator may be loaded by processor 292 to control the operation of the escape interval counters therein and to define refractory periods during which detection of R-waves and P-waves is ineffective to restart the escape interval counters.

If IMD 216 is configured to generate and deliver defibrillation shocks to heart 212, the signal generator may include a high voltage charge circuit and a high voltage output circuit. In the event that generation of a cardioversion or defibrillation shock is required, processor 292 may employ the escape interval counter to control timing of such cardioversion and defibrillation shocks, as well as associated refractory periods. In response to the detection of atrial or ventricular fibrillation or tachyarrhythmia requiring a cardioversion pulse, processor 292 may activate a cardioversion/defibrillation control module, which may be a hardware component of processor 292 and/or a firmware or software module executed by one or more hardware components of processor 292. The cardioversion/defibrillation control module may initiate charging of the high voltage capacitors of the high voltage charge circuit of the signal generator under control of a high voltage charging control line.

Processor 292 may monitor the voltage on the high voltage capacitor, e.g., via a voltage charging and potential (VCAP) line. In response to the voltage on the high voltage capacitor reaching a predetermined value set by processor 292, processor 292 may generate a logic signal that terminates charging. Thereafter, timing of the delivery of the defibrillation or cardioversion pulse by the signal generator is controlled by the cardioversion/defibrillation control module of processor 292. Following delivery of the fibrillation or tachycardia therapy, processor 292 may return the signal generator to a cardiac pacing function and await the next successive interrupt due to pacing or the occurrence of a sensed atrial or ventricular depolarization.

The signal generator may deliver cardioversion or defibrillation shocks with the aid of an output circuit that determines whether a monophasic or biphasic pulse is delivered, whether housing electrode 258 serves as cathode or anode, and which electrodes are involved in delivery of the cardioversion or defibrillation shocks. Such functionality may be provided by one or more switches or a switching module of the signal generator.

Telemetry circuit 14 is configured to receive downlink telemetry from and send uplink telemetry to programmer 224 with the aid of an antenna, which may be internal and/or external. Telemetry circuit 14 may perform modulation of uplink telemetry signals and demodulation of downlink telemetry signals. Telemetry circuit 14 may be controlled by processor 292. Telemetry circuit 14 includes any suitable hardware, firmware, software or any combination thereof for communicating with another device, such as programmer 224 (FIG. 11). Processor 292 may provide the data to be uplinked to programmer 224 and the control signals for the telemetry circuit within telemetry circuit 14, e.g., via an address/data bus. In some examples, telemetry circuit 14 may provide received data to processor 292 via a multiplexer.

In some examples, processor 292 may transmit atrial and ventricular heart signals (e.g., EGM signals) produced by atrial and ventricular sense amp circuits within electrical sensing module 298 to programmer 224. Programmer 224 may interrogate IMD 216 to receive the EGMs. Processor 292 may store EGMs within memory 294, and retrieve stored EGMs from memory 294. Processor 292 may also generate and store marker codes indicative of different cardiac events that electrical sensing module 298 detects, such as ventricular and atrial depolarizations, and transmit the marker codes to programmer 224. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

Telemetry circuit 14 is electrically coupled to telemetry antenna 12 via a telemetry conduction path formed by telemetry feedthrough assembly 90, protection circuit 92, impedance matching circuit 94 and conductors 96, 98, 10, 102.

Power source 278 is configured to supply power to one or more of the components within IMD 216. Power source 278 may include a rechargeable or non-rechargeable battery. A non-rechargeable battery may be capable of holding a charge for several years, while a rechargeable battery may be inductively charged from an external device, e.g., on a daily or weekly basis. Examples of a rechargeable battery include, but are not limited to, a lithium ion battery, a lithium polymer battery or a supercapacitor. Each of the components within IMD 216 may be electrically coupled to power source 278.

The techniques in this disclosure have been described with respect to examples that use a single stub filter. However, in other examples, a plurality of quarter-wave open-circuit stub filters may be electrically coupled to the telemetry conduction path in one or more locations. One or more of the plurality of stub filters may be tuned to attenuate different interfering frequencies. For example, a first stub filter may be configured to attenuate an interfering signal having a frequency of 45 megahertz (MHz) signal, e.g., an interfering signal produced by a 1.0 Tesla (T) MRI scanner, a second stub filter may be configured to attenuate an interfering signal having a frequency of 64 MHz, e.g., an interfering signal produced by a 1.5 T MRI scanner, and a third stub filter may be configured to attenuate an interfering signal having a frequency of 128 MHz, e.g., an interfering signal produced by a 3.0 T MRI scanner. In further examples, an implantable telemetry system may include any combination of the first, second, or third stub filters and/or any number of stub filters tuned to attenuate different frequencies altogether.

In additional examples, the plurality of stub filters may include at least two stub filters configured to attenuate the same frequency of interfering signal. The stub filters may be electrically coupled to the telemetry conduction path at the same or different locations. The second stub filter may be configured to attenuate residual frequency components of the interfering signal that may be contained in the filtered signal produced by the first stub filter.

The techniques of this disclosure may be implemented by an IMD that is configured to provide pacing therapy, and/or cardio-version shocks. In addition, the techniques in this disclosure may also be applied to other types of IMDs. For example, the techniques in this disclosure may be applied to neurostimulators, including deep brain stimulators, spinal cord stimulators, peripheral nerve stimulators, pelvic floor stimulators, gastro-intestinal stimulators, or the like.

The techniques described in this disclosure, including those attributed to image IMD 216, programmer 224, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), static RAM (SRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the following claims.

1. An implantable medical device comprising:
a telemetry conduction path comprising a first end electrically coupled to a telemetry antenna and a second end electrically coupled to a telemetry circuit disposed within a housing of the implantable medical device; and
a stub filter electrically coupled to the telemetry conduction path and configured to attenuate an interfering signal induced in the telemetry conduction path, the stub filter comprising a dielectric and a conductor disposed within the dielectric, the conductor comprising a first end electrically coupled to the telemetry conduction path and a second end configured in an open circuit configuration, the conductor having an electrical length approximately equal to one-quarter of the wavelength of the interfering signal when propagating through the stub filter.

2. The implantable medical device of claim 1, wherein the stub filter is further configured to cause a reflected version of the interfering signal to propagate through the conductor to attenuate the interfering signal.

3. The implantable medical device of claim 2, wherein the stub filter is further configured to cause the reflected version of the interfering signal to destructively interfere with the interfering signal to produce a filtered signal, the filtered signal being attenuated at a frequency of the interfering signal.

4. The implantable medical device of claim 2, wherein the reflected version of the interfering signal is approximately 180 degrees out-of-phase with the interfering signal at a junction where the stub filter and telemetry conduction path meet.

5. The implantable medical device of claim 1, wherein the stub filter is further configured to operate together with a radio frequency (RF) ground conductor as an open circuit terminated transmission line.

6. The implantable medical device of claim 1, wherein the interfering signal has a frequency of approximately 45 megahertz (MHz).

7. The implantable medical device of claim 1, wherein the interfering signal has a frequency of approximately 64 MHz.

8. The implantable medical device of claim 1, wherein the interfering signal has a frequency of approximately 128 MHz.

9. The implantable medical device of claim 1, wherein the interfering signal comprises at least one of a magnetic field, an electric field and an electromagnetic field generated by a magnetic resonance imaging (MRI) device.

10. The implantable medical device of claim 1, wherein the dielectric has a dielectric constant that is greater than or equal to 9000.

11. The implantable medical device of claim 1, wherein the conductor has a physical length of less than 0.5 inches.

12. The implantable medical device of claim 1, wherein the telemetry conduction path comprises a main conductor and a secondary conductor, wherein the first end of the first conductor of the stub filter is electrically coupled to the main conductor, and wherein the stub filter further comprises a second conductor electrically coupled to the secondary conductor of the telemetry conduction path, the second conductor of the stub filter being separated from the first conductor of the stub filter by the dielectric.

13. The implantable medical device of claim 1, wherein the stub filter is completely contained within a connector block of the implantable medical device and external to the housing of the implantable medical device.

14. The implantable medical device of claim 1, wherein the stub filter is completely contained within the housing of the implantable medical device.

15. The implantable medical device of claim 1, wherein the stub filter is further configured to allow a telemetry signal having a frequency between approximately 402 MHz and approximately 405 MHz to pass between the telemetry antenna and the telemetry circuit.

16. The implantable medical device of claim 1, wherein the stub filter is a first stub filter, wherein the interfering signal is a first interfering signal having a first frequency, and wherein the device further comprises:
a second stub filter electrically coupled to the telemetry conduction path, the second stub filter being configured to attenuate a second interfering signal induced in the telemetry conduction path, the second interfering signal having a second frequency different from the first frequency, the second stub filter comprising a dielectric and a conductor disposed within the dielectric, the conductor of the second stub filter comprising a first end electrically coupled to the telemetry conduction path and a second end configured in an open circuit configuration, the conductor of the second stub filter having an electrical length approximately equal to one-quarter of the wavelength of the second interfering signal when propagating through the second stub filter.

17. The implantable medical device of claim 16, wherein the first stub filter is electrically coupled to the telemetry conduction path at a first location, and wherein the second stub filter is electrically coupled to the telemetry conduction path at a second location different from the first location.

18. The implantable medical device of claim 1, wherein the stub filter is a first stub filter electrically coupled to the telemetry conduction path at a first location, and wherein the device further comprises:
a second stub filter electrically coupled to the telemetry conduction path at a second location different from the first location, the second stub filter being configured to attenuate the interfering signal induced in the telemetry conduction path, the second stub filter comprising a dielectric and a conductor disposed within the dielectric, the conductor of the second stub filter comprising a first end electrically coupled to the telemetry conduction path and a second end configured in an open circuit configuration, the conductor of the second stub filter having an electrical length approximately equal to one-quarter of the wavelength of the interfering signal when propagating through the second stub filter.

19. A method comprising:
attenuating, with a stub filter, an interfering signal induced in a telemetry conduction path comprising a first end electrically coupled to a telemetry antenna and a second end electrically coupled to a telemetry circuit disposed within a housing of the implantable medical device, the stub filter being electrically coupled to the telemetry conduction path, the stub filter comprising a dielectric and a conductor disposed within the dielectric, the conductor comprising a first end electrically coupled to the telemetry conduction path and a second end configured in an open circuit configuration, the conductor having an electrical length approximately equal to one-quarter of the wavelength of the interfering signal when propagating through the stub filter.

20. The method of claim 19, wherein attenuating the interfering signal comprises:
causing a reflected version of the interfering signal to propagate through the conductor to attenuate the interfering signal.

21. The method of claim 20, wherein attenuating the interfering signal further comprises:
causing the reflected version of the interfering signal to destructively interfere with the interfering signal to produce a filtered signal, the filtered signal being attenuated at a frequency of the interfering signal.

22. The method of claim 20, wherein the reflected version of the interfering signal is approximately 180 degrees out-of-phase with the interfering signal at a junction where the stub filter and telemetry conduction path meet.

23. The method of claim 19, wherein the interfering signal has a frequency of approximately 45 megahertz (MHz).

24. The method of claim 19, wherein the interfering signal has a frequency of approximately 64 MHz.

25. The method of claim 19, wherein the interfering signal has a frequency of approximately 128 MHz.

26. The method of claim 19, wherein the interfering signal comprises at least one of a magnetic field, an electric field and an electromagnetic field generated by a magnetic resonance imaging (MRI) method.

27. The method of claim 19, wherein the dielectric has a dielectric constant that is greater than or equal to 9000.

28. The method of claim 19, wherein the conductor has a physical length of less than 0.5 inches.

29. The method of claim 19, wherein the stub filter is completely contained within a connector block of the implantable medical device and external to the housing of the implantable medical device.

30. The method of claim 19, wherein the stub filter is completely contained within the housing of the implantable medical device.

31. The method of claim 19, wherein the stub filter is configured to allow a telemetry signal having a frequency between approximately 402 MHz and approximately 405 MHz to pass between the telemetry antenna and the telemetry circuit.

32. The method of claim 19, wherein the stub filter is a first stub filter, wherein the interfering signal is a first interfering signal having a first frequency, and wherein the method further comprises:
attenuating, with a second stub filter electrically coupled to the telemetry conduction path, a second interfering signal induced in the telemetry conduction path, the second interfering signal having a second frequency different from the first frequency, the second stub filter comprising a dielectric and a conductor disposed within the dielectric, the conductor of the second stub filter comprising a first end electrically coupled to the telemetry conduction path and a second end configured in an open circuit configuration, the conductor of the second stub filter having an electrical length approximately equal to one-quarter of the wavelength of the second interfering signal when propagating through the second stub filter.

33. The method of claim 32, wherein the first stub filter is electrically coupled to the telemetry conduction path at a first location, and wherein the second stub filter is electrically coupled to the telemetry conduction path at a second location different from the first location.

34. The method of claim 19, wherein the stub filter is a first stub filter electrically coupled to the telemetry conduction path at a first location, and wherein the method further comprises:
attenuating, with a second stub filter, the interfering signal, the second stub filter being electrically coupled to the telemetry conduction path at a second location different from the first location, the second stub filter comprising a dielectric and a conductor disposed within the dielectric, the conductor of the second stub filter comprising a first end electrically coupled to the telemetry conduction path and a second end configured in an open circuit configuration, the conductor of the second stub filter having an electrical length approximately equal to one-quarter of the wavelength of the interfering signal when propagating through the second stub filter.

35. An apparatus comprising:
a telemetry conduction path comprising a first end electrically coupled to a telemetry antenna and a second end electrically coupled to a telemetry circuit disposed within a housing of the implantable medical device; and
means for attenuating, with a stub filter electrically coupled to the telemetry conduction path, an interfering signal induced in the telemetry conduction path, the stub filter comprising a dielectric and a conductor disposed within the dielectric, the conductor comprising a first end electrically coupled to the telemetry conduction path and a second end configured in an open circuit configuration, the conductor having an electrical length approximately equal to one-quarter of the wavelength of the interfering signal when propagating through the stub filter.
